(19) United States
(12) Patent Application Publication
(10) Pub. No.: US 2024/0408027 A1
(43) Pub. Date: Dec. 12, 2024

(54) HIGH DRUG LOADING POLYMERS

(71) Applicant: Vanderbilt University, Nashville, TN (US)

(72) Inventors: Craig L. Duvall, Nashville, TN (US); Richard d'Arcy, Nashville, TN (US); Patrick Gerber, Nashville, TN (US); Marcus Bernard, Nashville, TN (US)

(21) Appl. No.: 18/698,763

(22) PCT Filed: Oct. 6, 2022
(86) PCT No.: PCT/US2022/045950

§ 371 Date: Apr. 4, 2024

Related U.S. Application Data
(60) Provisional application No. 63/252,966, filed on Oct. 6, 2021.

(51) Int. Cl. A61K 9/50; A61K 31/337; A61K 31/4418; A61K 31/4706; A61K 31/4745; A61K 31/506; A61K 31/517; A61K 31/573; A61K 31/69; A61K 31/7048; A61K 38/13; C08G 81/02

(52) U.S. Cl. CPC A61K 9/5031; A61K 31/337; A61K 31/4418; A61K 31/4706; A61K 31/4745; A61K 31/506; A61K 31/517; A61K 31/573; A61K 31/69; A61K 31/7048; A61K 38/13; C08G 81/025

(57) ABSTRACT

Disclosed herein arm block copolymers that have beneficial drug loading properties. An example block copolymer includes a first block that is hydrophilic and a second block having pendant groups that can non-covalently interact with a drug. Also disclosed are particles including self-assembled block copolymers and a drug; and methods of treating diseases.

### CROSS-REFERENCE TO RELATED APPLICATIONS

This application claims priority to U.S. Provisional Patent Application No. 63/252,966 filed on Oct. 6, 2021, which is incorporated fully herein by reference.

### TECHNICAL FIELD

The present disclosure relates to polymers designed to improve drug loading capacity and their use in biomedical applications, such as drug delivery.

### INTRODUCTION

Water insoluble drugs, such as paclitaxel and GANT58, are potential drug candidates for treatment in a number of different diseases. However, due to their water insolubility, they yield low in vivo bioavailability. Prior attempts to improve delivery of these drug candidates have resulted in carriers with low drug loading and/or immune response stimulating activity. Accordingly, there remains a need for polymers that provide high drug loading and efficient drug delivery.

### SUMMARY

In one aspect, disclosed are block copolymers including a first block (A), wherein the first block comprises a hydrophilic polymer; and a second block (B), wherein the second block comprises recurring units of formula (I)

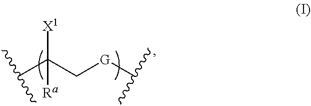

wherein:
X¹ is of formula (II)

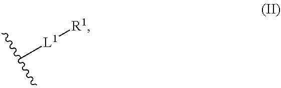

or
formula (III)

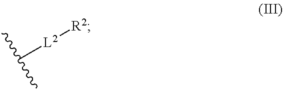

L¹ and L² are each independently —C(O)O-alkylene, —C(O)NR^{b}-alkylene, alkylene, alkylene-O-alkylene, alkylene-O—, or bond; R¹ is a π-interacting moiety; R² is a hydrogen donating moiety; R^{a} is hydrogen or C₁₋₃alkyl; R^{b} is hydrogen or C₁₋₆alkyl; and G is S, O, or bond.

In another aspect, disclosed are compositions including a plurality of block copolymers as disclosed herein self-assembled into a particle; and a drug encapsulated within the particle.

In another aspect disclosed are methods of treating a disease or disorder in a subject in need thereof, the method including administering to the subject a therapeutically effective amount of a composition as disclosed herein.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 is a schematic showing an example conjugation of N₃-PDMA (A Block) with Bz/OH polysulfide dialkyne core (B Block).

FIG. 2 is a schematic showing an example synthesis of N₃-poly(dimethyl acrylamide)(PDMA) (A Block).

FIG. 3 is a schematic showing an example anionic ring-opening polymerization of benzyl and (protected)alcohol thiiranes from difunctional initiator and end-capping via propargyl bromide (B block).

FIG. 4 is a schematic showing an example synthesis of benzyl and (protected) alcohol thiirane monomers from epoxides using thiourea as sulfur donor.

FIG. 5 is a schematic showing an example deprotection of alcohol groups.

FIG. 6 is a plot showing the effect of H₂O₂ on drug release (Nile Red used as surrogate drug) from example particles.

FIG. 7 is a plot showing serum stability of example particles (at 0.1 mg/mL) loaded with fluorescence resonance energy transfer (FRET) pair acceptor dye (DiI) and donor dye (DiO) and exposed to 50% serum.

FIG. 8 is a schematic showing an example particle preparation by thin-film rehydration from chloroform/chloroform-methanol solvent mixtures into deionized water with vigorous stirring at 50° C. followed by sonication bath.

FIG. 9 is a schematic showing example drug and polymer interactions via π-π interactions and hydrogen bonding.

FIG. 10 is a series of plots showing loading efficiency and capacity of model drugs into example benzyl(Bz)hydroxyl(OH)-polysulfide series (30-100% Bz) and comparison with model drug carrier surfactants (Pluronic F127, PEG-PLGA and PEG-PLA). FIG. 10A—cyclosporin A; FIG. 10B—paclitaxel; FIG. 10C—bortezomib; FIG. 10D—etoposide; FIG. 10E—neratinib; FIG. 10F—osimertinib; FIG. 10G—chloroquine; and FIG. 10F—GANT58. “Bz” is also denoted as “B” within this figure and other figures.

FIG. 11 is a series of plots showing loading and co-loading of paclitaxel and GANT58 into example Bz:OH polysulfide polymers (co-loading in 80% Bz). FIG. 11A shows loading efficiency and capacity for GANT58 into example particles. FIG. 11B shows loading efficiency and capacity for paclitaxel into example particles. FIG. 11C shows loading capacity of GANT58 and paclitaxel into example particles.

FIG. 12 is a transmission electron microscopy (TEM) image of example paclitaxel loaded nanoparticles (80% Bz). Scale bar=100 nm. Example particles here for 100-30% Bz—were ˜170-210 nm by DLS.

FIG. 13 are plots showing the maximum tolerated dose of paclitaxel in 80% Bz (FIG. 13A) or 40% Bz (FIG. 13B) formulated nanoparticles.

FIG. 14 is a plot showing pharmacokinetics of Cy5-labelled, paclitaxel-loaded example nanoparticles (80% Bz formulation) when injected at 15, 30, 60 or 125 mg of paclitaxel per kg of mouse weight.

FIG. 15 are plots showing therapeutic outcomes of 40% Bz (‘Fast’ release) and 80% Bz (‘Slow’ release) example paclitaxel-loaded nanoparticles when tested at their MTD of 150 mg per kg of mouse body weight in an orthotopic model of breast cancer (MDA-MB-231s in mammary fat pad of nude mice). Formulations were benchmarked against Taxol at its MTD (20 mg/kg). FIG. 15A, FIG. 15B, and FIG. 15C indicate only a single dose of treatment whereas FIG. 15D, FIG. 15E, FIG. 15F, and FIG. 15G (an expanded view of FIG. 15D) indicate a multiple dosing regimen (every 10 days, q10d).

FIG. 16 are plots and images showing BALB/c females intratibially injected with 4T1 bone-clone cells and treated 2× per week with saline or example 80% Bz NPs loaded with GANT58 (75 mg/kg) or PTX (PTX@75 mg/kg or PTX@150 mg/kg). FIG. 16A & FIG. 16B: X-rays were used to estimate bone lesion burden based on lesion area and greyscale analysis. Data presented as mean±SEM. Two-way ANOVA followed by a Dunnett's post-hoc analysis against saline control was performed. FIG. 16C: microCT (μCT) was used to determine trabecular bone volume fraction (BV/TV). Stats: two-way ANOVA with Tukey's post-hoc analysis. FIG. 16D: 3D rendering of trabeculae was performed to visualize representative examples of TIBD. FIG. 16E. Tumor burden was quantified via histology (H&E) from 5 mm longitudinally cut sections of tibia. FIG. 16F. representative images. In A-C, n=up to 8, *; P≤0.05, **; P≤0.01, ***; P≤0.001.

### DETAILED DESCRIPTION

### 1. Definitions

Unless otherwise defined, all technical and scientific terms used herein have the same meaning as commonly understood by one of ordinary skill in the art. In case of conflict, the present document, including definitions, will control. The materials, methods, and examples disclosed herein are illustrative only and not intended to be limiting. Methods and materials similar or equivalent to those described herein can be used in practice or testing of the disclosed invention. All publications, patent applications, patents and other references mentioned herein are incorporated by reference in their entirety.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The term “alkoxy” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy and tert-butoxy.

The term “alkoxyalkyl” as used herein, refers to an alkoxy group, as defined herein, appended to the parent molecular moiety through an alkylene group, as defined herein.

The term “alkyl,” as used herein, refers to a straight or branched, saturated hydrocarbon chain containing from 1 to 20 carbon atoms. The term “lower alkyl” or “C₁-C₆ alkyl” means a straight or branched chain hydrocarbon containing from 1 to 6 carbon atoms. The term “C₁-C₄ alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl and n-dodecyl.

The term “alkylene,” as used herein, refers to a divalent group derived from a straight or branched chain hydrocarbon of 1 to 50 carbon atoms, for example, of 2 to 10 carbon atoms. Representative examples of alkylene include, but are not limited to, —CH₂CH₂—, —CH₂CH₂CH₂—, —CH₂CH₂CH₂CH₂—, and —CH₂CH₂CH₂CH₂CH₂—.

The term “aryl,” as used herein, refers to a phenyl group, or a bicyclic or tricyclic fused ring system in which at least one ring is aromatic. Examples of bicyclic fused ring systems include a phenyl group appended to the parent molecular moiety and fused to another phenyl group, a cycloalkyl group, a heteroaryl group, or a heterocyclic group. Representative examples of aryl include, but are not limited to, phenyl, naphthyl, anthracenyl, indolyl, and tetrahydroquinolinyl.

The term “chemically protected,” as used herein in the conventional chemical sense and pertains to one or more reactive functional groups of a compound being protected from undesirable chemical reactions under specified conditions (e.g., pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts; 3rd Edition; John Wiley and Sons, 1999).

The term “cycloalkoxy,” as used herein, refers to a cycloalkyl group, as defined herein, appended to the parent molecular moiety through an oxygen atom.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl). Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “carbocyclyl” means a “cycloalkyl” or a “cycloalkenyl.” The term “carbocycle” means a “cycloalkane” or a “cycloalkene.” The term “carbocyclyl” refers to a “carbocycle” when present as a substituent.

The term “heteroalkylene” as used herein, means an alkylene group, as defined herein, in which one or more of the carbon atoms has been replaced by a heteroatom selected from S, Si, O, P and N.

The term “heteroaryl” as used herein, refers to an aromatic monocyclic ring or an aromatic bicyclic ring system or an aromatic tricyclic ring system. The aromatic monocyclic rings are five or six membered rings containing at least one heteroatom independently selected from the group consisting of N, O and S (e.g. 1, 2, 3, or 4 heteroatoms independently selected from O, S, and N). The five membered aromatic monocyclic rings have two double bonds and the six membered aromatic monocyclic rings have three double bonds. The bicyclic heteroaryl groups are exemplified by a monocyclic heteroaryl ring appended to the parent molecular moiety and fused to a monocyclic cycloalkyl group, as defined herein, a monocyclic aryl group, as defined herein, a monocyclic heteroaryl group, as defined herein, or a monocyclic heterocycle, as defined herein. The tricyclic heteroaryl groups are exemplified by a monocyclic heteroaryl ring appended to the parent molecular moiety and fused to two of a monocyclic cycloalkyl group, as defined herein, a monocyclic aryl group, as defined herein, a monocyclic heteroaryl group, as defined herein, or a monocyclic heterocycle, as defined herein. Representative examples of monocyclic heteroaryl include, but are not limited to, pyridinyl (including pyridin-2-yl, pyridin-3-yl, pyridin-4-yl), pyrimidinyl, pyrazinyl, thienyl, furyl, thiazolyl, thiadiazolyl, isoxazolyl, pyrazolyl, and 2-oxo-1,2-dihydropyridinyl. Representative examples of bicyclic heteroaryl include, but are not limited to, chromenyl, benzothienyl, benzodioxolyl, benzotriazolyl, quinolinyl, thienopyrrolyl, thienothienyl, imidazothiazolyl, benzothiazolyl, benzofuranyl, indolyl, quinolinyl, imidazopyridine, benzooxadiazolyl, and benzopyrazolyl. Representative examples of tricyclic heteroaryl include, but are not limited to, dibenzofuranyl and dibenzothienyl. The monocyclic, bicyclic, and tricyclic heteroaryls are connected to the parent molecular moiety through any carbon atom or any nitrogen atom contained within the rings, and can be unsubstituted or substituted.

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “hydroxyl” or “hydroxy,” as used herein, means an —OH group.

The term “hydroxyalkyl,” as used herein, means at least one —OH group, is appended to the parent molecular moiety through an alkylene group, as defined herein.

The term “substituted” refers to a group that may be further substituted with one or more non-hydrogen substituent groups. Substituent groups include, but are not limited to, halogen, =O (oxo), =S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

Terms such as “alkyl,” “cycloalkyl,” “alkylene,” etc. may be preceded by a designation indicating the number of atoms present in the group in a particular instance (e.g., “C₁₋₄alkyl,” “C₁₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,”the members of the group that follows may have any number of carbon atoms falling within the recited range. A “C₁₋₄alkyl,” for example, is an alkyl group having from 1 to 4 carbon atoms, however arranged (i.e., straight chain or branched).

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

The term “drug” refers to a substance that can act on a cell, virus, tissue, organ, organism, or the like, to create a change in the functioning of the cell, virus, tissue, organ, or organism. Examples of drugs include, but are not limited to, peptide-based drugs, chemotherapeutics, anti-inflammatory drugs, and immunomodulating drugs. A drug is capable of treating and/or ameliorating a condition or disease, or one or more symptoms thereof, in a subject. Drugs of the present disclosure also include prodrug forms of the agent.

The term “effective amount” or “therapeutically effective amount” refers to an amount sufficient to effect beneficial or desirable biological and/or clinical results.

The terms “subject” or “subject in need thereof,” as used herein, refer to a target of administration, which optionally displays symptoms related to a particular disease, pathological condition, disorder, or the like. The subject of the herein disclosed methods can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

The terms “treatment” or “treating” refer to the medical management of a patient with the intent to heal, cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

### 2. Block Copolymers

Provided herein are block copolymers having structures that can take advantage of non-covalent interactions with a drug to improve its loading properties. The block copolymer can include two different polymer blocks—the first block can be referred to as the A block and the second block can be referred to as the B block. The two different blocks can differ in hydrophilicity and/or hydrophobicity, which can allow the copolymer to self-assemble into different particulate structures.

The block copolymer can be synthesized in a variety of different structures. For example, the block copolymer can be an A-B diblock copolymer, an A-B-A triblock copolymer, a B-A-B triblock copolymer, or a star multiblock copolymer. In some embodiments, the block copolymer is an A-B diblock copolymer or an A-B-A triblock copolymer. Different copolymer structures can facilitate the formation of different particulate structures.

The block copolymer can have a varying molecular weight. For example, the block copolymer can have a number average molecular weight of about 1 kilodalton (kDa) to about 100 kDa, such as about 2 kDa to about 75 kDa, about 3 kDa to about 60 kDa, about 4 kDa to about 50 kDa, about 5 kDa to about 45 kDa, about 1 kDa to about 50 kDa, about 1 kDa to about 30 kDa, about 1 kDa to about 25 kDa, or about 1 kDa to about 15 kDa. In some embodiments, the block copolymer has a number average molecular weight of greater than 1 kDa, greater than 2 kDa, greater than 3 kDa, greater than 4 kDa, greater than 5 kDa, greater than 10 kDa, or greater than 20 kDa. In some embodiments, the block copolymer has a number average molecular weight of less than 100 kDa, less than 75 kDa, less than 60 kDa, less than 50 kDa, less than 40 kDa, less than 30 kDa, or less than 20 kDa.

Molecular weight of the block copolymer and the individual blocks can be measured by techniques used within the art, such as size exclusion chromatography (SEC), SEC combined with multi-angle light scattering, gel permeation chromatography, intrinsic viscosity, and the like.

The block copolymer can have a varying structure associated with a varying molecular weight. For example, the block copolymer can have a structure of A_{0.5 kDa-40 kDa}-B_{1 kDa-50 kDa}, A_{0.5 kDa-40 kDa}-B_{1 kDa-50 kDa}-A_{0.5 kDa-40 kDa}, A_{0.5 kDa-40 kDa}-B_{1 kDa-10 kDa}, or A_{0.5 kDa-10 kDa}-B_{1 kDa-10 kDa}-A_{0.5 kDa-10 kDa}. In some embodiments, the block copolymer has a structure of A_{4 kDa}-B_{3 kDa}. In some embodiments, the block copolymer has a structure of A_{4 kDa}-B_{3 kDa}-A_{4 kDa}. The foregoing are example structures and any combination of molecular weight and block alignment as disclosed herein can be used in the block copolymers.

### A. First Block (A)

The first block of the copolymer is a hydrophilic block. Accordingly, the first block can include a hydrophilic polymer. The first block can include one type of hydrophilic polymer or can include a plurality of different hydrophilic polymers. For example, in some embodiments, the first block includes at least 2, at least 3, at least 4, or at least 5 different hydrophilic polymers. Different hydrophilic polymers can be included within the same A block or each different hydrophilic polymer can correspond to a different A block within the block copolymer. The first block may act as a hydrophilic corona block (e.g., in particulate form). The first block may also instill stealth properties (e.g., evading a subject's immune system and/or clearance from the blood stream) to particles formed by the block copolymer.

Any suitable hydrophilic polymer known within the art can be used such that the block copolymer can, e.g., self-assemble into particulate structures. Example hydrophilic polymers include, but are not limited to, poly(dimethylacrylamidexPDMA), poly(ethylene glycol) (PEG), poly(PEG), poly(methyl oxazoline)(PMOX), poly(ethyl oxazoline), polysarcosine, poly(4-acryloylmorpholine), poly(glycerol monomethacrylate), poly(propylene sulfoxide), poly(2-(methylsulfinyl)ethyl acrylatexPMSEA), poly(vinyl alcohol)(PVA), poly(glycidol), poly(thioglycidyl glycerol)(PTGG), poly(2-methacryloyloxyethyl phosphorylcholine)(PMPC), poly(vinyl pyrrolidone)(PVP), poly(N-(2-hydroxypropyl)methacrylamide)(PHPMA), poly(trimethylamine N-oxide), poly(lysine-methacrylamide), poly(lysine-acrylamide), poly(carboxybetaine), poly(sulfobetaine), Heparosan, and any combination thereof.

In some embodiments, the first block includes PDMA, PEG, poly(methyl oxazoline), polysarcosine, poly(4-acryloylmorpholine), poly(glycerol monomethacrylate), poly(propylene sulfoxide), or a combination thereof. In some embodiments, the first block includes PDMA, PEG, poly(glycerol monomethacrylate), or a combination thereof. In some embodiments, the first block includes PDMA.

The first block can have a varying molecular weight. For example, the first block can have a number average molecular weight of about 0.5 kDa to about 40 kDa, such as about 1 kDa to about 30 kDa, about 2 kDa to about 25 kDa, about 3 kDa to about 20 kDa, about 4 kDa to about 15 kDa, about 0.5 kDa to about 25 kDa, or about 1 kDa to about 20 kDa. In some embodiments, the first block has a number average molecular weight of greater than 0.5 kDa, greater than 1 kDa, greater than 2 kDa, greater than 3 kDa, greater than 4 kDa, greater than 5 kDa, or greater than 10 kDa. In some embodiments, the first block has a number average molecular weight of less than 40 kDa, less than 35 kDa, less than 30 kDa, less than 25 kDa, less than 20 kDa, less than 15 kDa, or less than 10 kDa. In some embodiments, the first block has a number average molecular weight of about 0.5 kDa to about 5 kDa.

### B. Second Block (B)

The second block is more hydrophobic than the first block. The second block can include polymer(s) having a core monomer group (e.g., making up the polymer backbone) and a pendant group(s) attached to the core monomer group. The polymer(s) can include two different pendant groups that can aid in drug loading properties, such as loading capacity and loading efficiency. The two pendent groups can include a hydrogen bond donating moiety and a π-interacting moiety. The pendent groups can be included in the second block in a random manner and can be included in varying amounts.

Hydrogen bond donating moieties can allow the second block and copolymer thereof to participate in hydrogen bonding interactions with, e.g., a drug. As used herein, “hydrogen bonding” refers to a primarily electrostatic force of attraction between a hydrogen atom which is covalently bound to a more electronegative “donor” atom or moiety, and another electronegative atom bearing a lone pair of electrons, which can be called the hydrogen bond acceptor moiety. For example, drugs having a hydrogen bond acceptor moiety, such as amides, pyridines, aldehydes, ketones, esters, carboxylic acids, sulfoxides, and sulfones, can participate in hydrogen bonding interactions with the hydrogen bond donating moiety of the second block. Example hydrogen bond donating moieties include, but are not limited to, hydroxyl, carboxy, amine, ammonium, amide, imide, hydroxylamine, hydrazine, hydrazide, phenol, aniline, urea, thiourea, sulfonylamine, acetamidine, oxime, carbamate, O-thiocarbamate, S-thiocarbmate, and boronic acid. The second block can include the same hydrogen bond donating moiety throughout the second block. In other embodiments, the second block includes at least 2, at least 3, at least 4, or at least 5 different hydrogen bond donating moieties throughout the second block. In some embodiments, the hydrogen bond donating moiety includes hydroxyl, carboxy, amine, amide, or a combination thereof. In some embodiments, the hydrogen bond donating moiety includes hydroxyl, amine, or a combination thereof. In some embodiments, the hydrogen bond donating moiety includes hydroxyl.

π-interacting moieties can allow the second block and copolymer thereof to participate in π-π-interactions with, e.g., a drug. As used herein, “π-π-interactions” refer to a particular type of dispersion force from, e.g., van der Waals forces, which can be established between unsaturated (poly)cyclic moieties. For example, drugs having a π-interacting moiety, such as aromatic moieties, can participate in π-π interactions with the π-interacting moiety of the second block. Example π-interacting moieties include, but are not limited to, phenyl, naphthyl, pyridinyl, pyrrole, imidazolyl, pyrazolyl, indolyl, and furyl—all of which can be optionally substituted. The second block can include the same π-interacting moiety throughout the second block. In other embodiments, the second block includes at least 2, at least 3, at least 4, or at least 5 different n-interacting moieties throughout the second block. In some embodiments, the π-interacting moiety includes phenyl, furyl, pyridinyl, pyrrolyl, imidazolyl, or a combination thereof. In some embodiments, the π-interacting moiety includes phenyl, furyl, pyridinyl, or a combination thereof. In some embodiments, the π-interacting moiety includes phenyl.

The pendant groups can be included in the second block as a copolymer. For example, the pendant groups can be introduced into the second block through different monomer populations. In some embodiments, the pendent groups are included as a random copolymer, an alternating copolymer, or a block copolymer. In some embodiments, the pendant groups are included as a random copolymer.

The polymer(s) of the second block can have varying core monomer groups. Core monomer groups can include any monomer having the above-listed pendant groups and that can be polymerized through chain-growth polymerization, such as anionic ring opening polymerization and free radical polymerization (e.g., atom transfer radical polymerization (ATRP)). Example core monomer groups include, but are not limited to, sulfide (e.g., thioether), acrylate, acrylamide, methacrylate, methacrylamide, and styrene. The second block can include the same core monomer group throughout the second block. In other embodiments, the second block includes at least 2, at least 3, at least 4, or at least 5 different core monomer groups throughout the second block. In some embodiments, the core monomer group of the second block includes sulfide, acrylate, acrylamide, methacrylate, methacrylamide, or a combination thereof. In some embodiments, the core monomer group of the second block includes sulfide, acrylate, methacrylate, or a combination thereof. In some embodiments, the core monomer group of the second block includes sulfide.

In embodiments that include a sulfide-based polymer, a reactive oxygen species-based release of a drug can be used, e.g., in particulate formulations. See general scheme below:

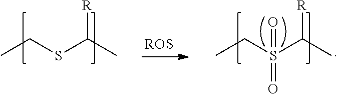

In addition, the R group (e.g., pendant group) can further be used to modulate the release of the drug. For example, it has been found that a higher concentration of a hydrogen bond donating moiety (e.g., hydroxyl), rather than a π-interacting moiety (e.g., phenyl) can provide a more rapid release of the drug in response to ROS. This may allow for a more beneficial dosing regimen in the methods of treatment discussed below. Overall, these properties can allow for a controlled release of the drug.

In some embodiments, the second block includes recurring units of formula (I)

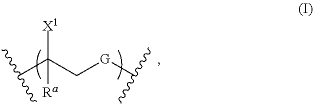

wherein: X¹ is of formula (II)

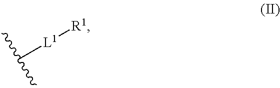

or
formula (III)

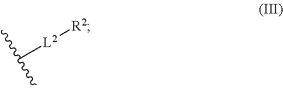

L¹ and L² are each independently —C(O)O-alkylene, —C(O)NR^{b}-alkylene, alkylene, alkylene-O-alkylene, alkylene-O—, or bond; R¹ is a π-interacting moiety; R² is a hydrogen bond donating moiety; R⁸ is hydrogen or C₁₋₃alkyl; R^{b} is hydrogen or C₁₋₆alkyl; and G is S, O, or bond.

In some embodiments, R¹ is an aryl or heteroaryl, wherein the aryl and heteroaryl are optionally substituted with 1 or 2 substituents, each independently halogen, cyano, C₁₋₄alkyl, C₁₋₂fluoroalkyl, —OC₁₋₂alkyl, or —OC₁₋₂fluoroalkyl; and R² is hydroxy, amine, or amide.

In some embodiments, R¹ is an aryl or heteroaryl, wherein the aryl and heteroaryl are optionally substituted with 1 or 2 substituents, each independently halogen, C₁₋₄alkyl, or —OC₁₋₂alkyl; and R² is hydroxy or amine.

In some embodiments, L¹ and L² are each independently —C(O)O—C₁₋₄alkylene, —C(O)NH—C₁₋₄alkylene, C₁₋₁₀alkylene, C₁₋₆alkylene-O—C₁₋₆alkylene, C₁₋₄alkylene-O—, or bond.

In some embodiments, L¹ and L² are each independently —C(O)O—C₁₋₂alkylene, —C(O)NH—C₁₋₂alkylene, C₁₋₆alkylene, C₁₋₄alkylene-O—C₁₋₄alkylene, C₁₋₂alkylene-O—, or bond.

In some embodiments, L¹ and L² are each independently C₁₋₁₀alkylene, C₁₋₆alkylene-O—C₁₋₆alkylene, or bond.

In some embodiments, L¹ and L² are each independently C₁₋₁₀alkylene or C₁₋₆alkylene-O—C₁₋₆alkylene; R¹ is aryl or heteroaryl; R² is hydroxy; and G is S or O.

In some embodiments, L¹ is C₁₋₄alkylene or C₁₋₂alkylene-O—C₁₋₂alkylene; L² is C₁₋₄ alkylene; R^{a} is hydrogen; and G is S.

In some embodiments, X¹ is of formula (II-a)

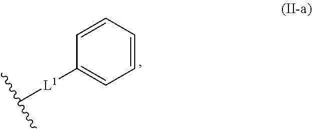

or
formula (III-a)

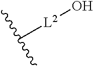

(III-a), wherein L¹ and L² are defined as described above for formula (I).

In some embodiments, X¹ is of formula (II-a)

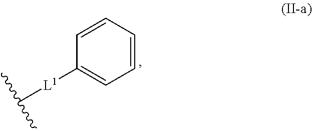

or
formula (III-a)

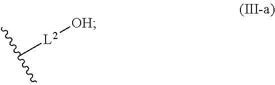

L¹ is C₁₋₄alkylene or C₁₋₂alkylene-O—C₁₋₂alkylene; L² is C₁₋₄alkylene; R^{a} is hydrogen; and G is S.

In some embodiments, the second block includes recurring units of formula (I-a)

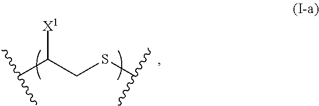

wherein X¹ is defined as described above in formula (I).

In some embodiments, the second block includes recurring units of formula (I-a)

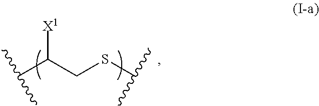

wherein: X¹ is of formula (II-a)

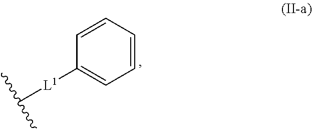

or
formula (III-a)

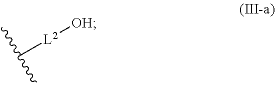

L¹ is C₁₋₄alkylene or C₁₋₂alkylene-O—C₁₋₂alkylene; and L² is C₁₋₄alkylene.

The formulas of (II) and (III) can be included in varying amounts within the second block. For example, the second block can include recurring units including formula (II) and recurring units including formula (M) at a weight ratio of about 100:0 to about 0:100 (formula (II):formula(III)), such as about 100:0 to about 30:70 (formula (II):formula(III)), about 90:10 to about 10:90 (formula (II):formula(III)); about 80:20 to about 20:80 (formula (II):formula(l)), about 70:30 to about 30:70 (formula (II):formula(II)), or about 60:40 to about 40:60 (formula (II):formula(III)). In some embodiments, the second block includes recurring units including formula (II) and recurring units including formula (III) at a weight ratio of about 100:0 (formula (II):formula(III)); about 90:10 (formula (II):formula(III)), about 80:20 (formula (I):formula(II)), about 70:30 (formula (II):formula(III)), about 60:40 (formula (II):formula(III)), about 50:50 (formula (II):formula(III)), about 40:60 (formula (II):formula(III)), about 30:70 (formula (II):formula(III)), about 20:80 (formula (II):formula(III)), or about 10:90 (formula (II):formula(III)).

Varying amounts of recurring units including formula (II) can be included in the second block. For example, the second block can include recurring units including formula (II) at about 30% to about 100% by weight of the second block, such as about 35% to about 95% by weight of the second block, about 40% to about 90% by weight of the second block, about 50% to about 100% by weight of the second block, about 50% to about 99% by weight of the second block, or about 60% to about 95% by weight of the second block.

In some embodiments, the second block includes recurring units including formula (U) at greater than 30% by weight of the second block, greater than 35% by weight of the second block, greater than 40% by weight of the second block, greater than 45% by weight of the second block, greater than 50% by weight of the second block, greater than 55% by weight of the second block, greater than 60% by weight of the second block, greater than 65% by weight of the second block, or greater than 70% by weight of the second block. In some embodiments, the second block includes recurring units including formula (II) at less than 100% by weight of the second block, less than 99% by weight of the second block, less than 98% by weight of the second block, less than 97% by weight of the second block, less than 96% by weight of the second block, less than 95% by weight of the second block, less than 90% by weight of the second block, less than 85% by weight of the second block, or less than 80% by weight of the second block.

Varying amounts of recurring units including formula (III) can be included in the second block. For example, the second block can include recurring units including formula (III) at about 0% to about 100% by weight of the second block, such as about 5% to about 90% by weight of the second block, about 5% to about 80% by weight of the second block, about 10% to about 70% by weight of the second block, about 1% to about 65% by weight of the second block, or about 10% to about 60% by weight of the second block.

In some embodiments, the second block includes recurring units including formula (III) at greater than 0% by weight of the second block, greater than 1% by weight of the second block, greater than 2% by weight of the second block, greater than 3% by weight of the second block, greater than 4% by weight of the second block, greater than 5% by weight of the second block, greater than 10% by weight of the second block, greater than 15% by weight of the second block, or greater than 20% by weight of the second block. In some embodiments, the second block includes recurring units including formula (III) at less than 100% by weight of the second block, less than 95% by weight of the second block, less than 90% by weight of the second block, less than 85% by weight of the second block, less than 80% by weight of the second block, less than 75% by weight of the second block, less than 70% by weight of the second block, less than 65% by weight of the second block, or less than 60% by weight of the second block.

The above description for formulas (II) and (III) with respect to amounts included in the second block can also be applied to formulas of (II-a) and (III-a).

In some embodiments, the recurring unit of formula (I) is selected from the group consisting of:

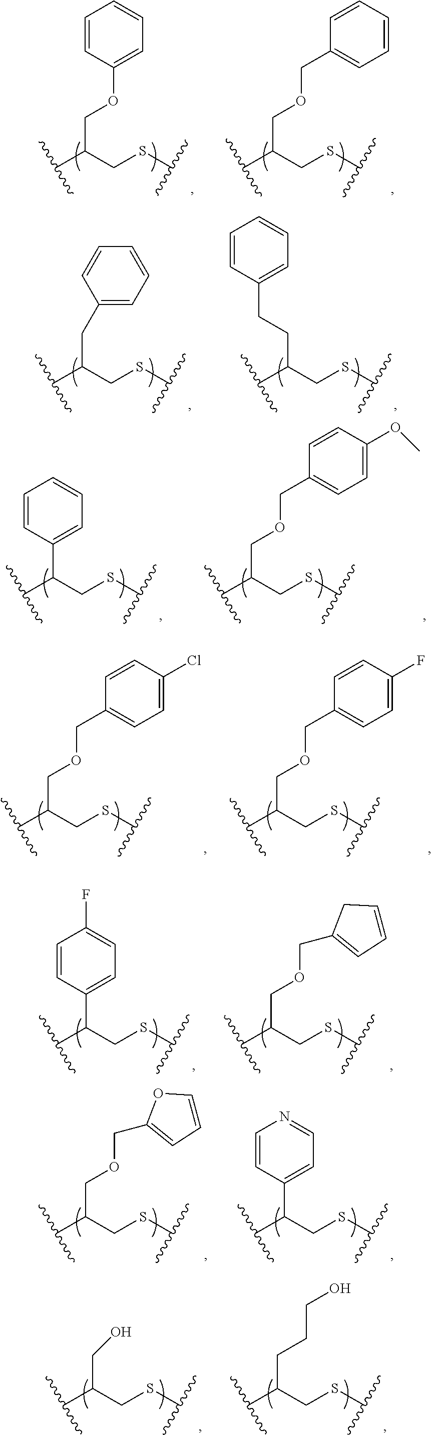

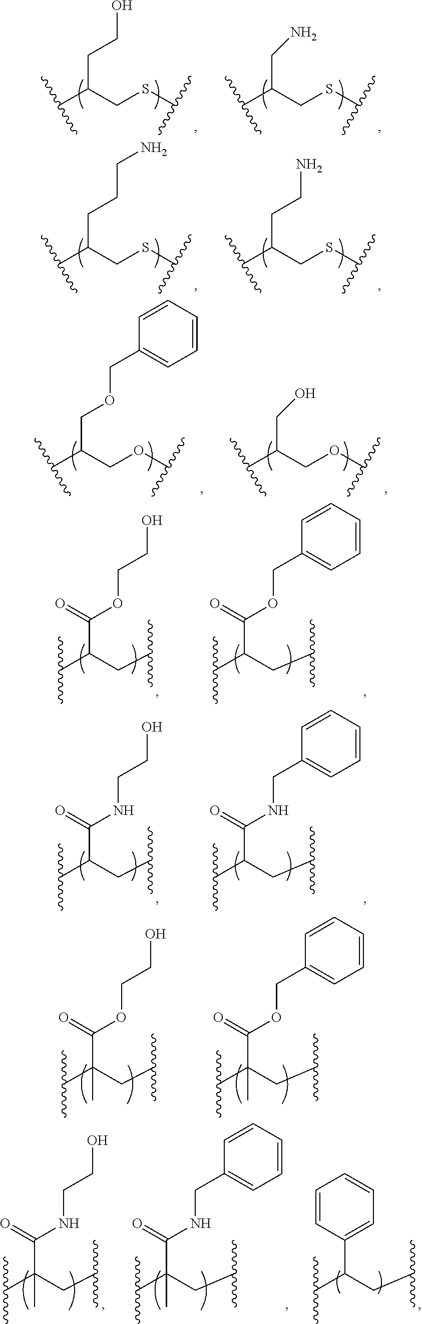

and a combination thereof.

In some embodiments, the recurring unit of formula (I) is selected from the group consisting of:

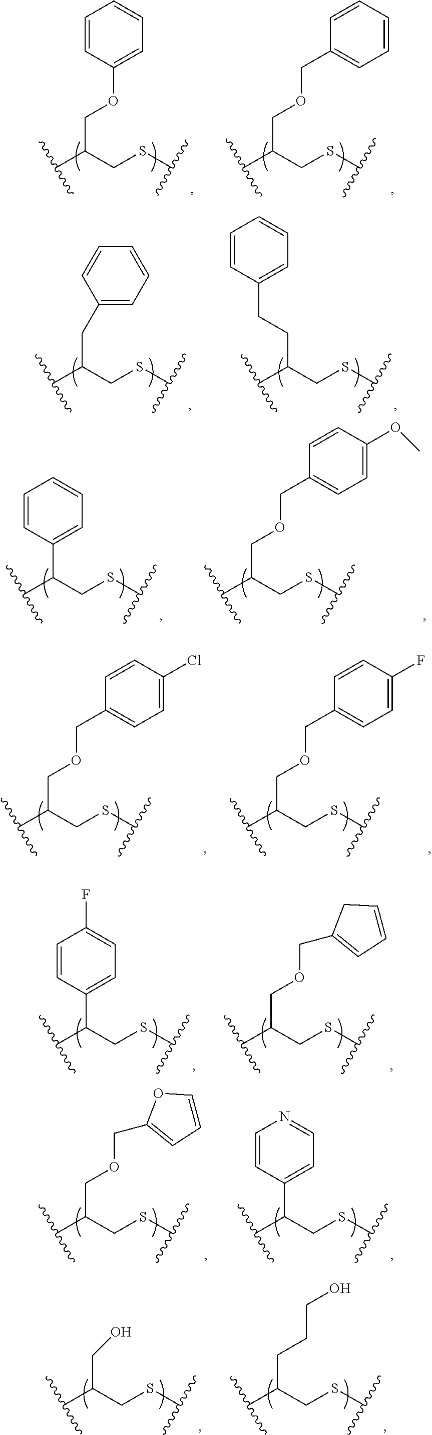

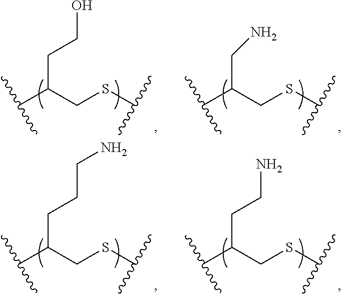

and a combination thereof.

In some embodiments, the second block includes recurring units of formula (IV)

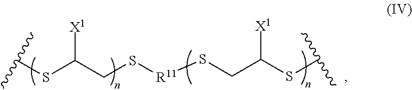

wherein: X¹ is defined as described above in formula (I); R¹¹ is alkylene or heteroalkylene, wherein the alkylene and heteroalkylene are optionally substituted; and n is 2 to 500.

In some embodiments, R¹¹ is alkylene or heteroalkylene; and n is 2 to 350.

In some embodiments, R¹¹ is C₁₋₁₅alkylene or C₁₋₁₅heteroalkylene, wherein 1, 2, or 3 carbon atoms of the heteroalkylene are replaced by a heteroatom selected from O and N.

In some embodiments, R¹¹ is C₁₋₁₀alkylene or C₁₋₁₀heteroalkylene, wherein 1, 2, or 3 carbon atoms of the heteroalkylene are replaced by O.

In some embodiments, R¹¹ is C₁₋₁₀alkylene or C₁₋₁₀heteroalkylene, wherein 1, 2, or 3 carbon atoms of the heteroalkylene are replaced by 0; and n is 2 to 350.

Each of the recurring units of the second block (e.g., recurring units of formula (I) and recurring units that fall under formula (I)) can be repeated a number of times. For example, a recurring unit of the second block can be repeated 2 to 500 times, such as 5 to 500 times, 5 to 400 times, 5 to 350 times, 10 to 300 times, 15 to 350 times, 5 to 200 times, 10 to 150 times, 5 to 400 times, or 5 to 275 times. In some embodiments, the recurring unit of the second block can be repeated greater than 5 times, greater than 6 times, greater than 7 times, greater than 8 times, greater than 9 times, greater than 10 times, greater than 15 times, or greater than 20 times. In some embodiments, the recurring unit of the second block can be repeated less than 500 times, less than 450 times, less than 400 times, less than 350 times, less than 325 times, less than 315 times, less than 300 times, or less than 250 times. The number of repeats of the recurring unit can also be expressed as a subscript “n” associated with the recurring unit as typically done in the art with polymers. For example, formula (I) can be denoted as follows

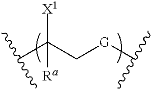

wherein n can be as described above, e.g., 5 to 500. All of the disclosed recurring units of formula (I) and those that fall under formula (I) can be denoted in the same manner. In addition, the recurring units can be repeated randomly throughout the second block, in series, or a combination thereof (e.g., some random and some in series within the second block). In addition, the recurring units can be attached to each other through a linker group (e.g., see R¹¹ of formula (IV)).

The second block can have a varying molecular weight. For example, the second block can have a number average molecular weight of about 1 kDa to about 50 kDa, such as about 2 kDa to about 45 kDa, about 3 kDa to about 40 kDa, about 4 kDa to about 40 kDa, about 5 kDa to about 35 kDa, about 6 kDa to about 30 kDa, about 7 kDa to about 25 kDa, about 1 kDa to about 25 kDa, about 1 kDa to about 15 kDa, or about 2 kDa to about 20 kDa. In some embodiments, the second block has a number average molecular weight of greater than 1 kDa, greater than 2 kDa, greater than 3 kDa, greater than 4 kDa, greater than 5 kDa, greater than 6 kDa, greater than 7 kDa, greater than 8 kDa, greater than 9 kDa, or greater than 10 kDa. In some embodiments, the second block has a number average molecular weight of less than 50 kDa, less than 45 kDa, less than 40 kDa, less than 35 kDa, less than 30 kDa, less than 25 kDa, less than 20 kDa, less than 15 kDa, or less than 10 kDa. In some embodiments, the second block has a number average molecular weight of about 1 kDa to about 6 kDa.

### C. Example Synthesis of the Second Block and Block Copolymers

Monomers used to provide recurring units of formula (I), wherein G is S, R^{a} is hydrogen, and X¹ is defined as described above for formula (I), may be prepared as shown in the general reaction scheme:

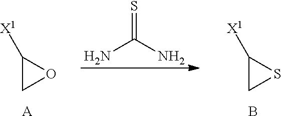

As shown in the general scheme above, X¹-substituted oxiranes of formula A may be reacted with thiourea under suitable conditions known to those of ordinary skill in the art to form the corresponding X¹-substituted thiiranes of formula B.

Thiiranes of formula B may subsequently be polymerized under suitable ring-opening polymerization conditions known to those of ordinary skill in the art and as shown in the general scheme below, wherein X¹, R¹¹, and n are defined as described above for formula (IV) to form recurring units of the second block. In addition, below describes a diblock initiator. Other initiators can also be used, such as mono- and multi-arm block initiators.

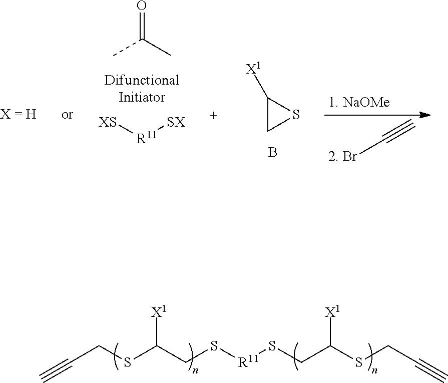

Anionic ring-opening polymerization of thiiranes of formula B can be reacted with a difunctional initiator and be end-capped via propargyl bromide to provide the second block. Any suitable end-capping group can be used in the synthesis of the second block, where the end capping group can be reactive with a functional group of the first block, directly end-capped with the first block, or the first block can be directly polymerized from this block (e.g. poly(thioglycidyl glycerol). Suitable difunctional initiators are known within the art. Example difunctional initiators include, but are not limited to, dithiols, dithiophenols, dithiolates, dialcoholates, xanthates, thiocarbamates, trithiocarbonates, and dithioacetates or otherwise protected thiols that can be deprotected and deprotonated into thiolates. Specific examples include, but are not limited to, 1,2-ethanedithiol, 1,3-propanedithiol, 1,3-butanedithiol, benzene-1,4-dithiol, 1,4-benzenedimethanethiol, 2,2′-(ethylenedioxy)diethanethiol, and 2,2′-(ethylenedioxy)diethanethioacetate.

In some embodiments, X¹ may be chemically protected, e.g., if X¹ has a terminal hydroxyl. Accordingly, in embodiments where X¹ is chemically protected, the synthesis can include a deprotection step. In some embodiments, the protecting groups can be removed under acidic conditions. For example, a suitable deprotection reagent is a Dowex ion-exchange resin.

The alkyne end-capped second block can then be reacted with the hydrophilic polymer of the first block through an azide via click chemistry techniques. For example, thiol-reactive chemistries such as maleimides, acrylates, methacrylates, acrylamides, methacrylamides, iodoacetamides, bromoacetamides, chloroacetamides, iodoacetates, bromoacetates, chloroacetates, alkyl-iodides, alkyl-bromides can be used to directly link to the second block after polymerization. Other click chemistry techniques include, but are not limited to, radical thiol-ene/thiol-yne reactions, Diels-Alder cycloadditions between tetrazines and strained alkenes or alkynes, condensation of amines/hydrazines/hydroxylamines with aldehyde/ketones or their amidation with activated esters, condensations of 1,2 aminothiols with 2-cyanobenzothiazoles, Pd-catalyzed cross-coupling between boronic esters and halobenzenes and strain promoted cycloaddition between strained alkynes and nitrones.

### D. Particles

The composition and structure of the block copolymer can afford the copolymers the ability to self-assemble into particulate structures. For example, the block copolymer can have a critical micelle concentration (CMC) and at a concentration above the CMC, the block copolymers can self-assemble, e.g., with other block copolymers into a particulate structure. Accordingly, also disclosed herein are particles that can include a plurality of self-assembled block copolymers. Example structures include micelles, vesicles, inverted micelles, spherical polymersomes, tubular polymersomes, and nanofibers. Micelles can be a variety of shapes with varied aspect ratios. For example, the particle can be a filomicelle, a rodlike micelle, a cylindrical micelle, and/or a wormlike micelle.

The particle can further include a drug, which is discussed in more detail below. The drug can be encapsulated within the particle. The drug can be located in different regions of the particle depending on the type of particle formed. For example, in a micelle, the first block can be a corona of the particle, while the second block can be a hydrophobic core of the particle, and the drug can be located within the core of the micelle. In vesicle embodiments, the plurality of block copolymers may form a bilayer and the drug can be located in the bilayer of the vesicle.

The block copolymer can have a varying CMC. For example, the block copolymer can have a CMC of about 0.01 mg/mL to about 0.07 mg/mL, such as about 0.015 mg/mL to about 0.06 mg/mL, about 0.02 mg/mL to about 0.055 mg/mL, about 0.025 mg/mL to about 0.055 mg/mL, or about 0.025 mg/mL to about 0.052 mg/mL. CMC can be determined by atomic force microscopy imaging analysis and/or fluorescence intensity analysis of micelle formation. Further details of determining CMC can be found in J. N. Phillips, Trans. Faraday 1955, 51, 561, which is incorporated by reference herein in its entirety.

The particle can have a varying diameter. For example, the particle can have a diameter of about 10 nm to about 500 nm, such as about 15 nm to about 400 nm, about 20 nm to about 300 nm, about 25 nm to about 200 nm, about 10 nm to about 100 nm, about 50 nm to about 500 nm, or about 10 nm to about 200 nm. Particle size can be measured by techniques known within the art, such as dynamic light scattering and electron microscopy (e.g., TEM).

### 3. Uses of Block Copolymers

### A. Compositions

Also disclosed herein are compositions that include a plurality of block copolymers self-assembled into a particle as described above. Accordingly, the description of the block copolymers, first block, second block, and particles above can be applied to the disclosed compositions. The composition can also include a drug encapsulated within the particle.

In contrast to current systems, which typically exploit H-bond acceptors, the disclosed polymers can uniquely leverage OH/H-bond donor combined with aromatic, as well as thioether π-electron interacting groups (e.g., in sulfide embodiments). S-π interactions are not extensivity explored in the drug delivery field but can be strong. Accordingly, in some embodiments, the polymers disclosed herein provide improved loading of H-bond accepting drugs. As such, the block copolymers can advantageously load drugs into a particulate form.

Any suitable drug can be used in the disclosed compositions. Drugs having a hydrogen bond acceptor moiety, aromatic moiety, or both can benefit in their loading into the particle as these moieties can beneficially interact with the second block of the disclosed copolymers. In some embodiments, the drug includes a hydrogen bond acceptor moiety, an aromatic moiety, or a combination thereof.

Drugs with a range of hydrophobicity can be used in the compositions. The drug's hydrophobicity may be characterized by its octanol-water distribution coefficient (log D), where a larger value indicates greater hydrophobicity. For example, the drug can have a log D of about −1 to about 10, such as about 0 to about 8, about 0.5 to about 7, about 1 to about 8, about 1 to about 7, about 0 to about 6, about 0.5 to about 5.5, or about 1 to about 5.5. In some embodiments, the drug has a log D of greater than −1, greater than −0.5, greater than 0, greater than 0.5, or greater than 1. In some embodiments, the drug has a log D of less than 10, less than 8, less than 7, less than 6, or less than 5.5. The above-listed log D values can be measured at a pH of 7.4 (e.g., log D of greater than 0.5 at a pH of 7.4).

In some embodiments, the drug includes a hydrogen bond acceptor moiety, an aromatic moiety, or a combination thereof, and has a log D of about −1 to about 10. The drug may also be in crystalline form in the particle.

Examples of drugs include, but are not limited to, peptide-based drugs (e.g., cyclosporin, bortezomib, etc.), chemotherapeutics (e.g., a taxane, a tyrosine kinase inhibitor, a topoisomerase inhibitor, a bcl-family inhibitor, a DNA crosslinking agent, a DNA antimetabolite, a PHD-family inhibitor, etc.); anti-inflammatory drugs (e.g., a steroid, a corticosteroid, a non-steroidal anti-inflammatory drug (NSAID), etc.); and immune modulating drugs (e.g., an immunotherapy adjuvant). In some embodiments, the drug includes a chemotherapeutic, an anti-inflammatory drug, an immune modulating drug, or a combination thereof. In some embodiments, the drug includes a taxane, a steroid, a corticosteroid, a NSAID, a tyrosine kinase inhibitor, a topoisomerase inhibitor, a bcl-family inhibitor, an immunotherapy adjuvants, a DNA crosslinking agent, a DNA antimetabolite, a PHD-family inhibitor, or a combination thereof.

In some embodiments, the drug includes cyclosporin A, paclitaxel, bortezomib, etoposide, neratinib, osimertinib, chloroquine, GANT58, docetaxel, dexamethasone, carmofur, dexamethasone, dexamethasone palmitate, carfilzomib, afatinib, irinotecan, doxorubicin, doxycycline, camptothecin, imiquimod, MK-8617, ciclopirox, roxadustat, or a combination thereof. In some embodiments, the drug includes cyclosporin A, paclitaxel, bortezomib, etoposide, neratinib, osimertinib, chloroquine, GANT58, or a combination thereof. In some embodiments, the drug includes paclitaxel, GANT 58, or a combination thereof.

As discussed elsewhere, the block copolymers can advantageously load drugs into a particulate form. For example, the particle can include the drug at about 0% to about 60% by weight of the particle, such as about 1% to about 60% by weight of the particle, about 5% to about 60% by weight of the particle, about 10⁰% to about 60% by weight of the particle, about 10% to about 55% by weight of the particle, about 15% to about 55% by weight of the particle, about 5% to about 55% by weight of the particle, about 20% to about 60% by weight of the particle, or about 30% to about 55% by weight of the particle. In some embodiments, the particle includes the drug at greater than 1% by weight of the particle, greater than 5% by weight of the particle, greater than 10% by weight of the particle, greater than 15% by weight of the particle, greater than 20% by weight of the particle, greater than 25% by weight of the particle, greater than 30% by weight of the particle, or greater than 35% by weight of the particle. In some embodiments, the particle includes the drug at less than 60% by weight of the particle, less than 55% by weight of the particle, less than 50% by weight of the particle, less than 45% by weight of the particle, less than 40% by weight of the particle, less than 35% by weight of the particle, or less than 30% by weight of the particle.

The particle can also load the drug with improved efficiency. Loading efficiency refers to the amount of drug encapsulated in the particle relative to the amount that was added to the particle. For example, the particle can load the drug at about 10% to about 1⁰⁰% efficiency, such as about 15% to about 90% efficiency, about 5% to about 80% efficiency, about 10% to about 70% efficiency, about 10% to about 60% efficiency, about 15% to about 55% efficiency, about 5% to about 55% efficiency, about 20% to about 80% efficiency, or about 100% to about 55% efficiency. In some embodiments, the particle includes the drug at greater than 1% efficiency, greater than 5% efficiency, greater than 10% efficiency, greater than 15% efficiency, greater than 200/a efficiency, greater than 25% efficiency, greater than 30% efficiency, or greater than 40% efficiency. In some embodiments, the particle includes the drug at less than 100% efficiency, less than 90% efficiency, less than 80% efficiency, less than 70% efficiency, less than 60% efficiency, less than 55% efficiency, or less than 50% efficiency.

i. Administration

The composition can be administered prophylactically or therapeutically. In prophylactic administration, the composition can be administered in an amount sufficient to induce a response. In therapeutic applications, the composition can be administered to a subject in need thereof in an amount sufficient to elicit a therapeutic effect. An amount adequate to accomplish this is defined as “therapeutically effective dose.” Amounts effective for this use will depend on, e.g., the particular composition of the conjugate regimen administered, the manner of administration, the stage and severity of the disease, the general state of health of the patient, and the judgment of the prescribing physician.

The composition may be presented in a single dose or as divided doses administered at appropriate intervals, for example, as two, three, four or more sub-doses per day. The sub-dose itself may be further divided, e.g., into a number of discrete loosely spaced administrations.

As will be readily apparent to one skilled in the art, the useful in vivo dosage to be administered and the particular mode of administration will vary depending upon the age, weight, the severity of the affliction, and subjects treated, the particular compounds employed, and the specific use for which these compounds are employed. The determination of effective dosage levels, that is the dosage levels necessary to achieve the desired result, can be accomplished by one skilled in the art using routine methods, for example, human clinical trials, in vivo studies and in vitro studies.

The compositions can be administered via a variety of routes. Typical delivery routes include parenteral administration, e.g., intradermal, intramuscular or subcutaneous delivery. Other routes include oral administration, intranasal, intravaginal, transdermal, intravenous, intraarterial, intratumoral, intraperitoneal, and epidermal routes. In some embodiments, the composition is administered intravenously, subcutaneously, intradermally, intramuscularly, or intraperitoneally. In some embodiments, the composition is administered intravenously.

Dosage amount and interval may be adjusted individually to provide plasma levels of the drug which are sufficient to maintain the modulating effects, or minimal effective concentration (MEC). The MEC will vary for each agent but can be estimated from in vivo and/or in vitro data. Dosages necessary to achieve the MEC will depend on individual characteristics and route of administration. However, assays well known to those in the art can be used to determine plasma concentrations. Dosage intervals can also be determined using MEC value. Compositions can be administered using a regimen which maintains plasma levels above the MEC for 10-90% of the time, such as between 30-90% or between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the symptoms to be treated and the route of administration. Further, the dose, and perhaps dose frequency, will also vary according to the age, body weight, and response of the individual patient. A program comparable to that discussed above may be used in veterinary medicine.

### B. Methods of Treating a Disease or Disorder

Further provided herein are methods of treating a disease or disorder in a subject in need thereof. The method can include administering to the subject a therapeutically effective amount of a composition as disclosed herein. Example diseases and disorders include, but are not limited to, cancer, inflammatory diseases, and wound healing. Example inflammatory diseases include, but are not limited to, osteoarthritis, rheumatoid arthritis, and ulcerative colitis. In some embodiments, the disease or disorder includes cancer, osteoarthritis, rheumatoid arthritis, ulcerative colitis, or wound healing. In some embodiments, the disease or disorder includes cancer or an inflammatory disease. In some embodiments, the disease or disorder includes cancer. In some embodiments, the disease or disorder is bone cancer or breast cancer. In some embodiments, the subject is human.

The disclosed compositions can provide numerous advantages in the treatment of diseases and disorders. For example, the composition can have a low level of drug leakage, e.g., from the particle, in serum. In some embodiments, the composition is stable in serum for at least 5 hours, at least 10 hours, at least 15 hours, at least 20 hours, at least 24 hours, at least 2 days, or at least 1 week. Stability in serum is indicated as having less than 30% drug leakage over the indicated time period. Stability can be measured by FRET as described in the Examples.

Due in part to the improved drug loading of the disclosed block copolymers and particles thereof, the disclosed compositions can have an increased maximum tolerated dose. For example, the composition can have a maximum tolerated dose of about 20 mg/kg to about 200 mg/kg, such as about 30 mg/kg to about 190 mg/kg, about 40 mg/kg to about 180 mg/kg, about 50 mg/kg to about 170 mg/kg, or about 100 mg/kg to about 200 mg/kg. In some embodiments, the composition has a maximum tolerated dose of at least 100 mg/kg, at least 110 mg/kg, at least 120 mg/kg, at least 130 mg/kg, at least 140 mg/kg, or at least 150 mg/kg. In some embodiments, the composition has a maximum tolerated dose of less than 200 mg/kg, less than 190 mg/kg, less than 180 mg/kg, less than 170 mg/kg, less than 160 mg/kg, or less than 150 mg/kg.

The compositions can also provide an improved treatment of the disease or disorder. For example, where the disease or disorder is cancer, the subject can have reduction in tumor volume of at least 20%/6, at least 30%, at least 50%, at least 60%, at least 70%, at least 80%, or at least 90% over a period of about 2 weeks to about 5 weeks following the beginning of a dosing regimen (e.g., composition administered 1× or 2× weekly). In some embodiments, the subject's tumor(s) is completely eradicated.

The description of the block copolymers, first block, second block, particles, compositions, and drug can also be applied to the methods of treatment disclosed herein.

The disclosed invention has multiple aspects, illustrated by the following non-limiting examples.

### 4. Examples

### Example 1

### Novel Polymer Library for Drug Delivery Applications

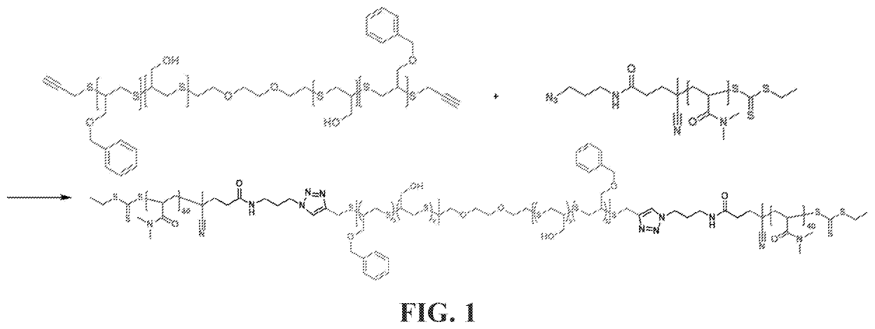

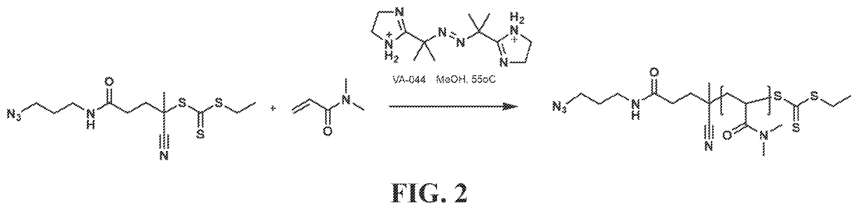

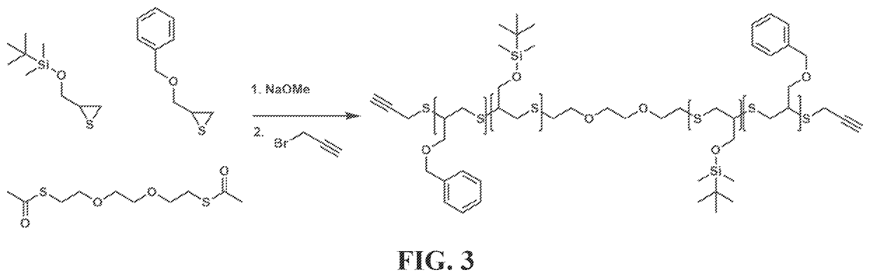

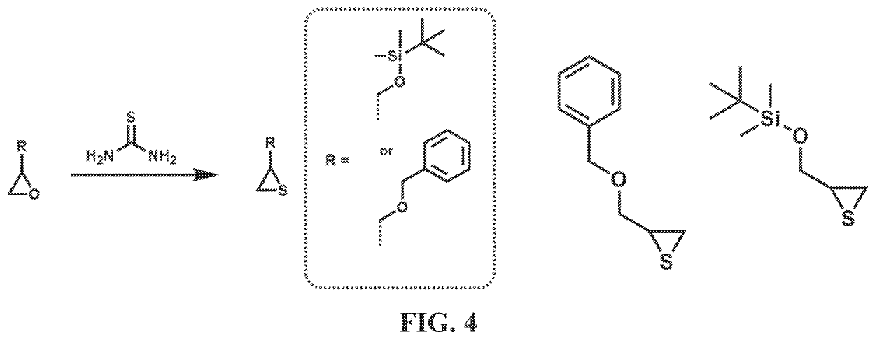

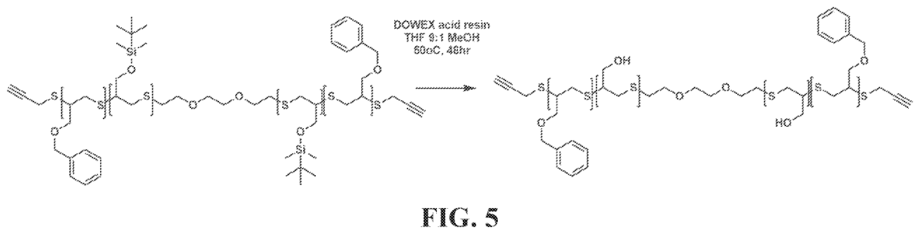

This example describes a novel polymer library capable of formulating certain drugs as nanoparticles at high drug-to-polymer loadings (high loading capacity) for drug delivery applications, such as anti-inflammatory or chemotherapeutic drugs. An example polymer is a A-B-A triblock-copolymers having the general structure of PDMA₄ₖ-b-P(Bzₓ/OH_{y})₃ₖ-b-PDMA₄ₖ (FIG. 1). Here, each A-block includes a 4 kDa poly(dimethylacrylamide)(PDMA) hydrophilic corona and is synthesized as illustrated in FIG. 2. These A-blocks provide aqueous stability to the nanoparticle and flank a statistical copolysulfide core (B-block), which contains a thioether backbone and a systematically varied wt. % of benzyl (Bz) and/or hydroxyl (OH) on the side chains (i.e., 100:0, 90:10, 80:20, 70:30, 60:40, 50:50, 40:60, 30:70—Bz:OH wt. %). Synthesis of the B-block includes anionic ring-opening co-polymerization of both benzylic- and (protected) OH-containing thiirane monomers (also known as episulfides, FIG. 3). The benzyl and (protected) alcohol thiirane monomers may be synthesized as shown in FIG. 4. Following co-polymerization, the resulting polysulfide may be deprotected as shown in FIG. 5. A molecular characterization of PBz/PBzTG copolymers is shown below in Table 1.

TABLE 1
Molecular Characterization of PBz/PBzTG Copolymers.

| | wt. % | Bz/OH | | Core |
|---|---|---|---|---|
| | Benzyl | mol ratio | | Mn (g/mol) |
| Core | (Bz) | ¹H NMR | Theor. | ¹H NMR |
| P(BzTG₁₆) | 100 | 18.2/0 | 3000 | 3281 |
| P(BzTG₁₅-TGG₃) | 90 | 16.8/3.4 | 3000 | 3335 |
| P(BzTG_{13.3}-TGG_{6.7}) | 80 | 13.6/6.7 | 3000 | 3055 |
| P(BzTG_{11.7}-TGG₁₀) | 70 | 12.9/10.4 | 3000 | 3261 |
| P(BzTG₁₀-TGG_{13.3}) | 60 | 11.1/14.6 | 3000 | 3315 |
| P(BzTG_{8.3}-TGG_{16.7}) | 50 | 9.8/17.7 | 3000 | 3360 |
| P(BzTG_{6.7}-TGG₂₀) | 40 | 6.8/19.0 | 3000 | 2936 |
| P(BzTG₅-TGG_{23.3}) | 30 | 4.8/20.4 | 3000 | 2701 |

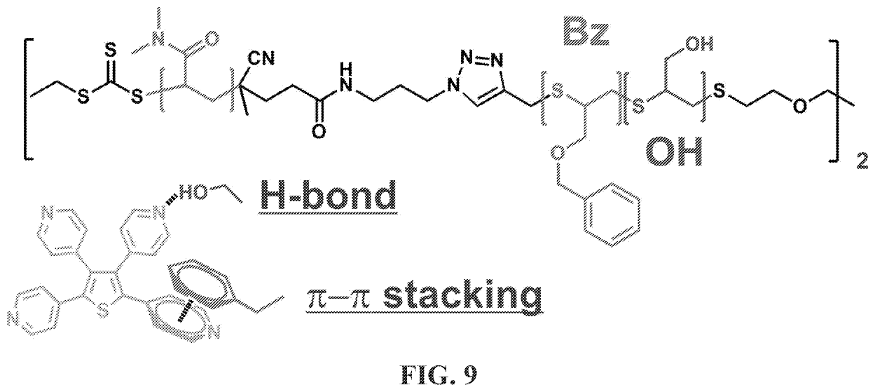

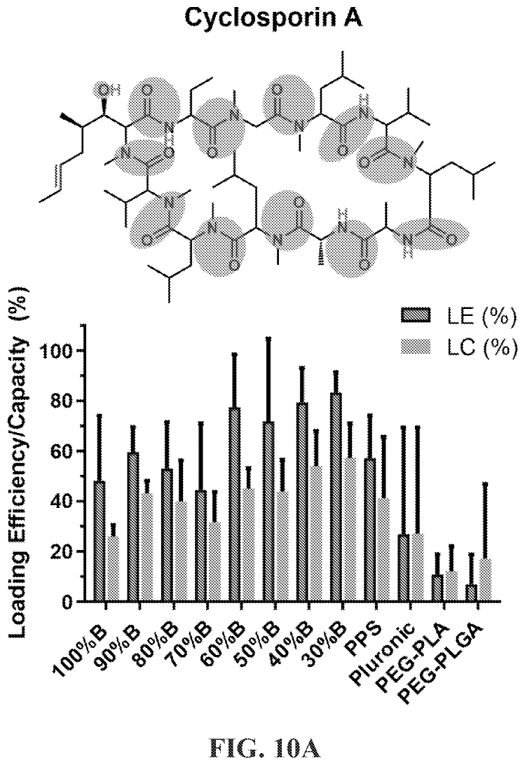

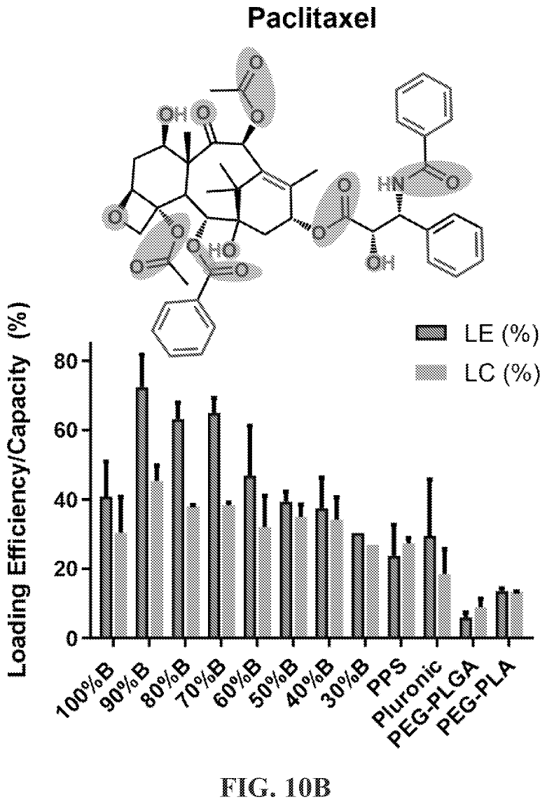

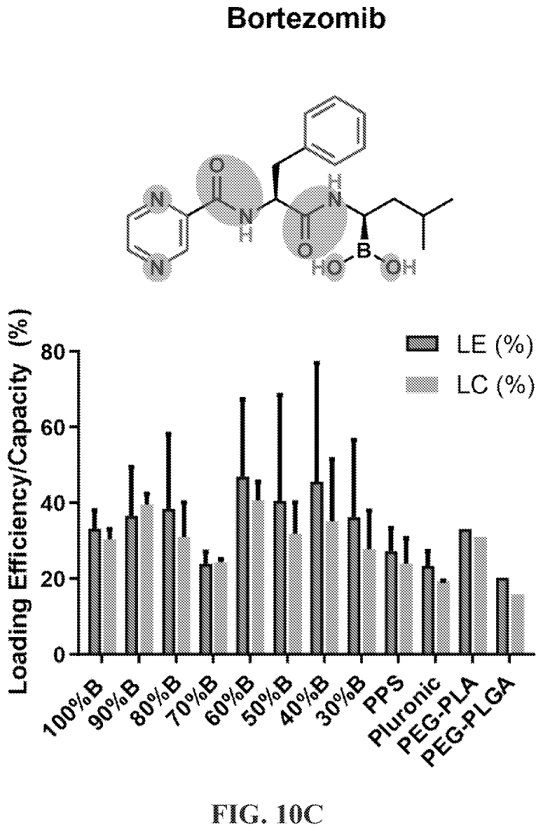

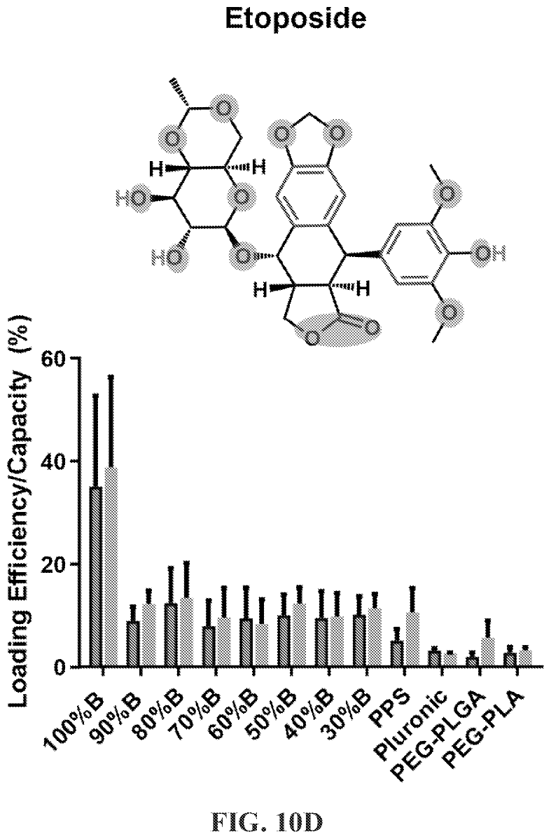

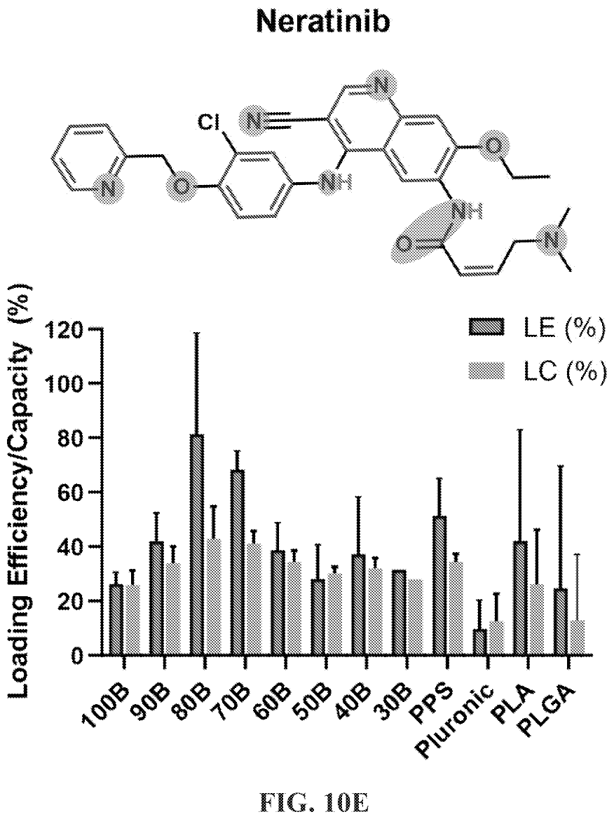

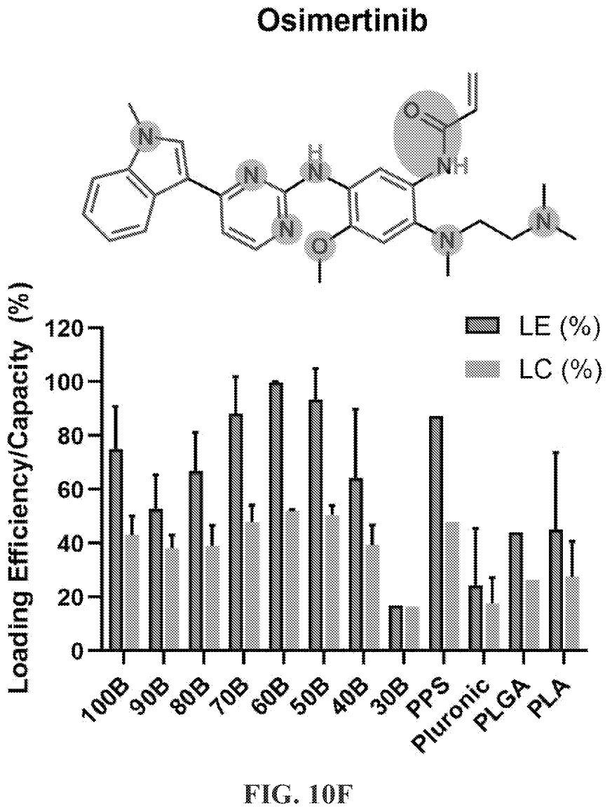

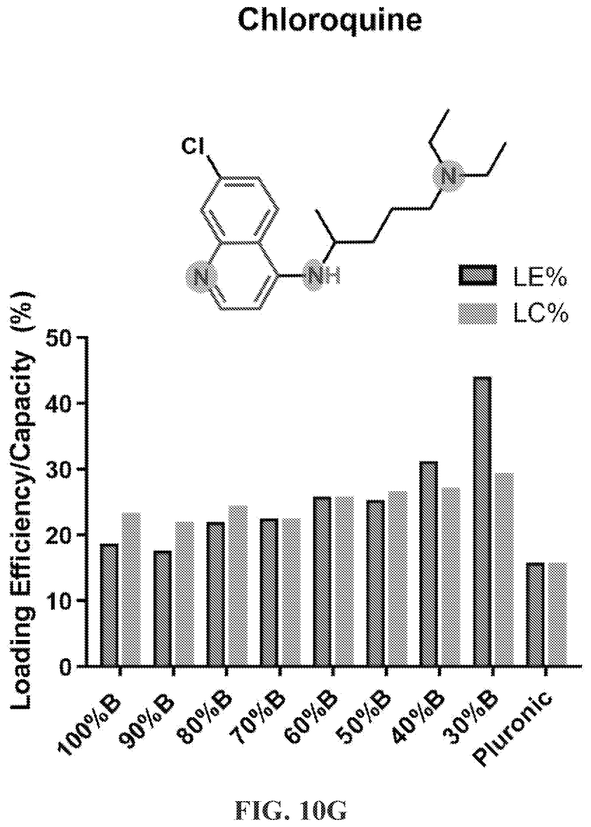

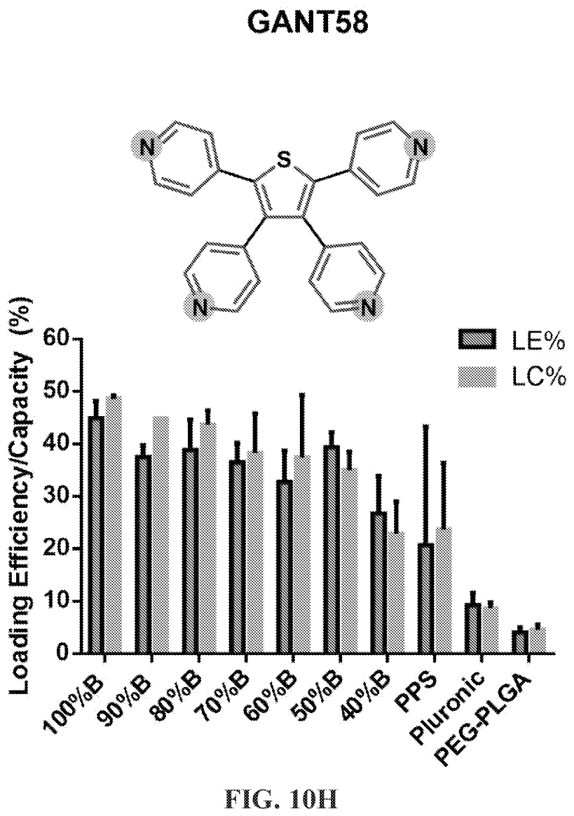

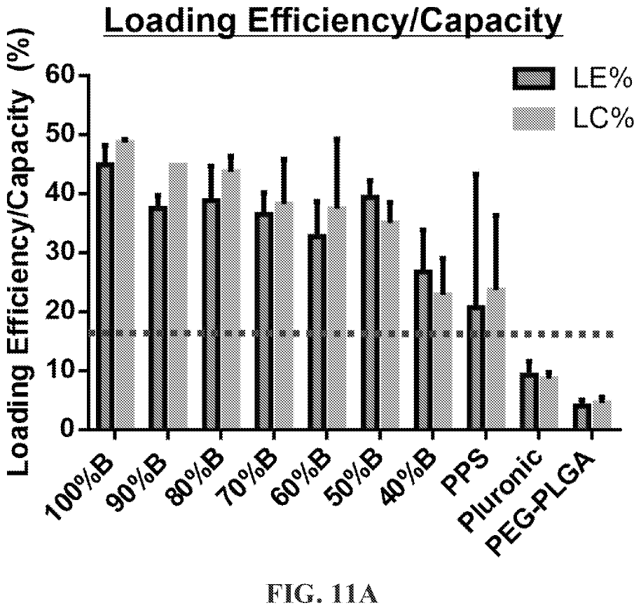

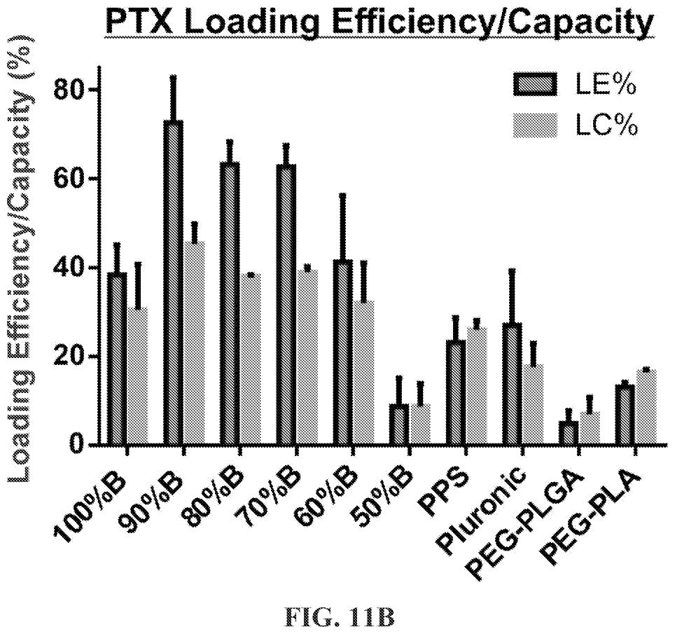

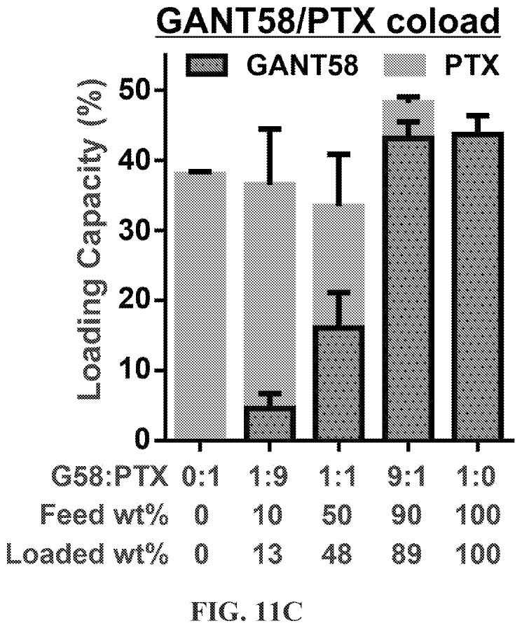

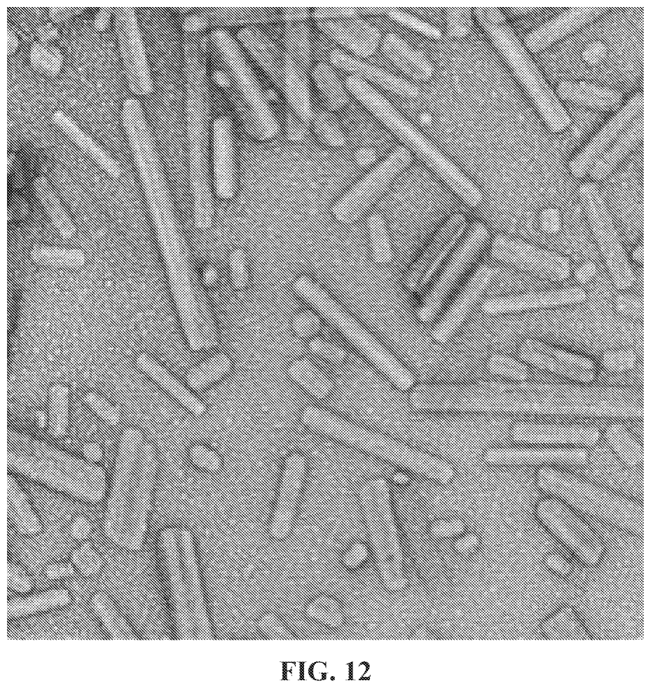

The core polysulfide permits (1) high drug loading and (2) an appropriate drug release. For achieving both of these points, maximizing drug-polymer interactions is useful. In this example, a polymeric library was produced by systematically modulating Bz:OH composition ratios of the core polysulfide and screening each polymer for its ability to load a range of chemically diverse drugs (FIGS. 10A-10H). As illustrated in FIG. 11A-C, the polymeric library exhibited ultrahigh loading of multiple drugs, as well as the ability to co-load and maintain ultrahigh loading capacity. In addition, some drugs were loaded into rod-like particles (FIG. 12). Drugs with a predominance of hydrogen(H)-bond acceptor and/or aromatic moieties such as peptide-based drugs were found to load extremely well, i.e. high-drug loading was achieved when i) H-bonding is effectively provided between H-bond acceptor group(s) of the drug(s) (e.g., amides, pyridines, aldehydes, ketones, esters, carboxylic acids, sulfoxides, sulfones) and H-bond donor OH moieties on the polysulfide side-chain, as well as between ii) aromatic moieties of the drug and side-chain Bz groups and/or thioethers groups of the polysulfide backbone to maximize π-π interactions and/or π-S interactions respectively. FIG. 9 shows an example of these contributing interactions between GANT58 and the polysulfides.

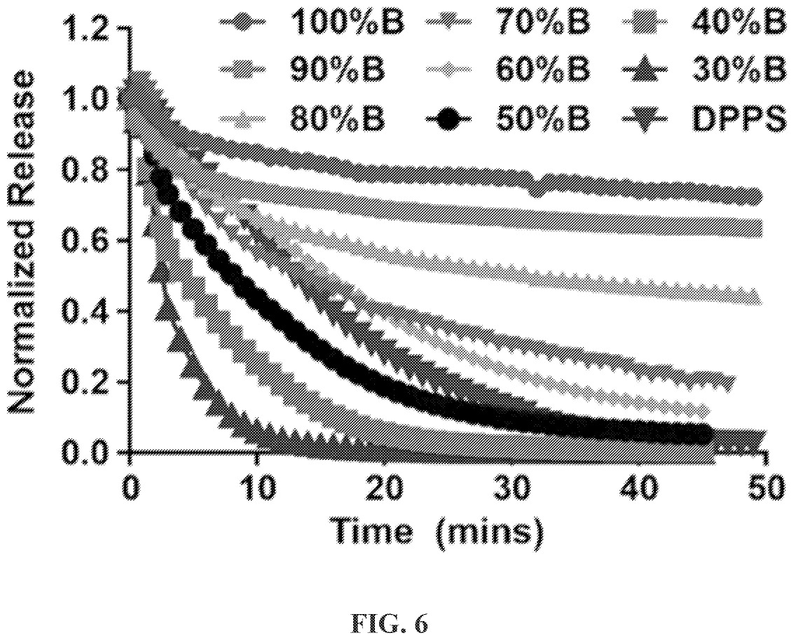

In regard to drug-release, the polysulfide cores are also biologically-responsive to reactive oxygen species (ROS) (e.g., release drug in response to ROS). As ROS are significantly elevated in inflamed and cancerous tissues, the biologically-responsive activity of the polysulfide cores allows for the specific or enhanced drug released in these environments. This permits a more targeted and faster release of drug at the diseased location. It has also been found that the ROS-stimulated release rate of loaded cargo was accelerated with increasing wt. % of OH in the polysulfide core (FIG. 6). The Bz:OH ratio therefore also enables tailoring of drug release rate, ranging from rapid (high OH:Bz) to slow release rates (low OH:Bz).

The polymers discussed herein were benchmarked against other commonly used polymers in drug delivery such as Pluronic F127, PEG-PLGA and PEG-PLA (highly analogous to the clinically approved Genexol PM formulation). In most examples, the present polymer series obtained a higher drug loading capacity and efficiency at an optimal Bz:OH ratio (FIG. 6). This is important in the context of drug (vehicle) efficacy, as higher loading capacities allow for high drug doses to be administered (high maximum tolerated doses, MTDs) and thus better treatment outcomes.

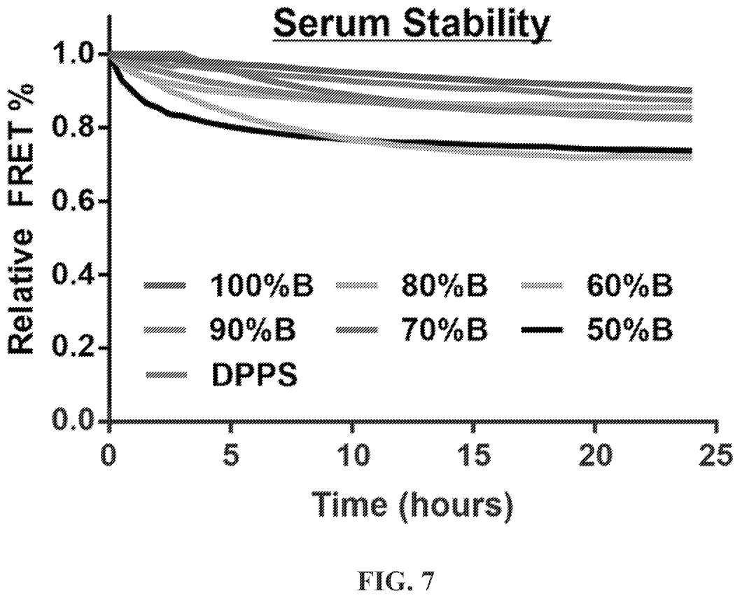

Toxicities in nanoparticles often arise from inherent toxicity/immunogenicity of the carrier itself as well as from premature and excessive drug leakage into the blood due to poor drug-polymer interaction and/or high critical micellization concentrations (CMCs). All members of the Bz:OH polymer library were determined to have an appropriately low CMC value (typically between 0.02-0.05 mg/mL) and very low drug leakage in serum (<20% in 24 hours for the 100 70% Bz formulations, and ˜30% in the 60% or less Bz formulations, FIG. 7).

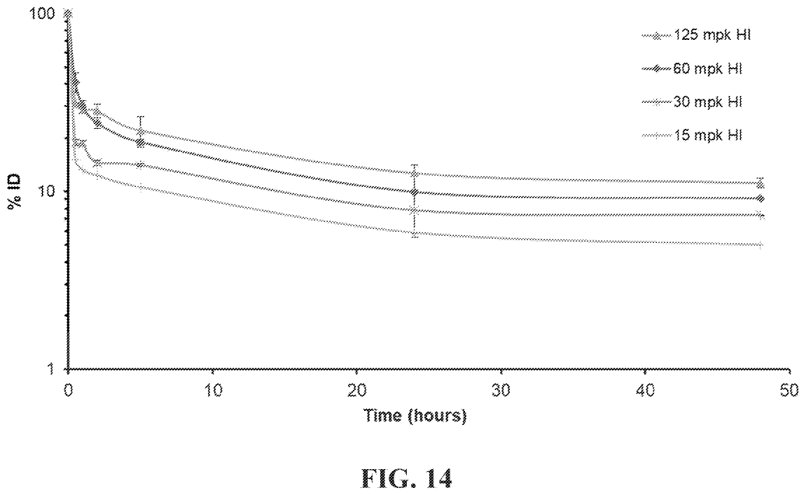

In addition, shown in FIG. 14 is the pharmacokinetics of Cy5-labeleed paclitaxel loaded nanoparticles (80% Bz formulation) injected at various dosages.

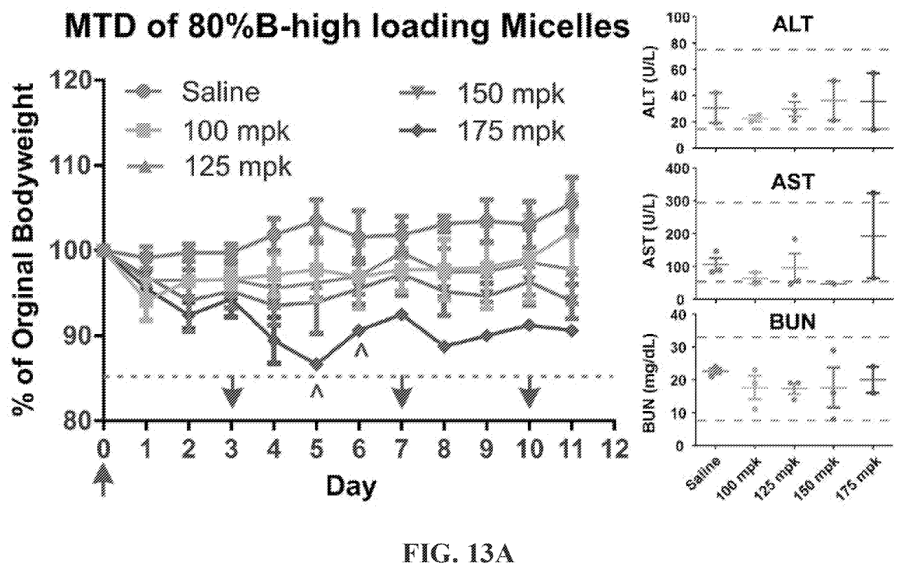

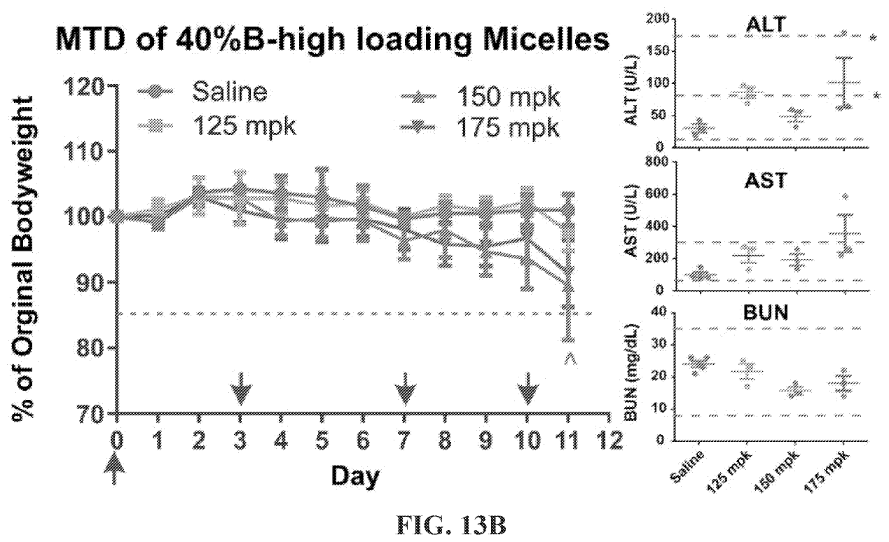

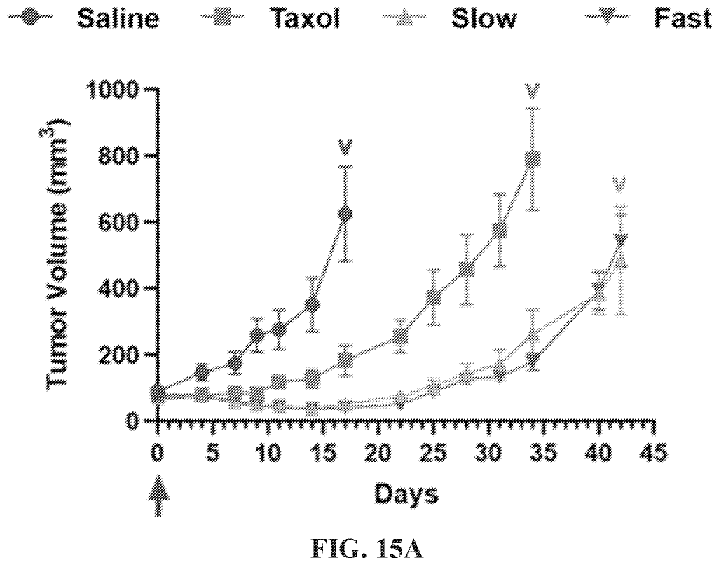

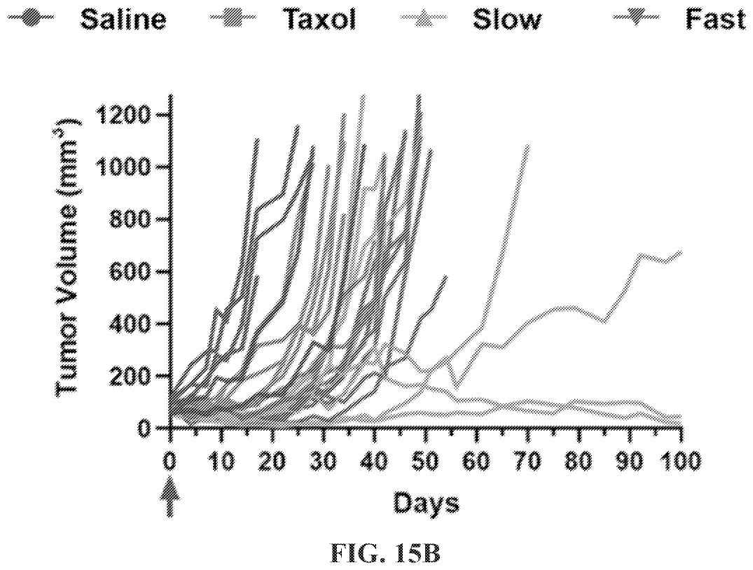

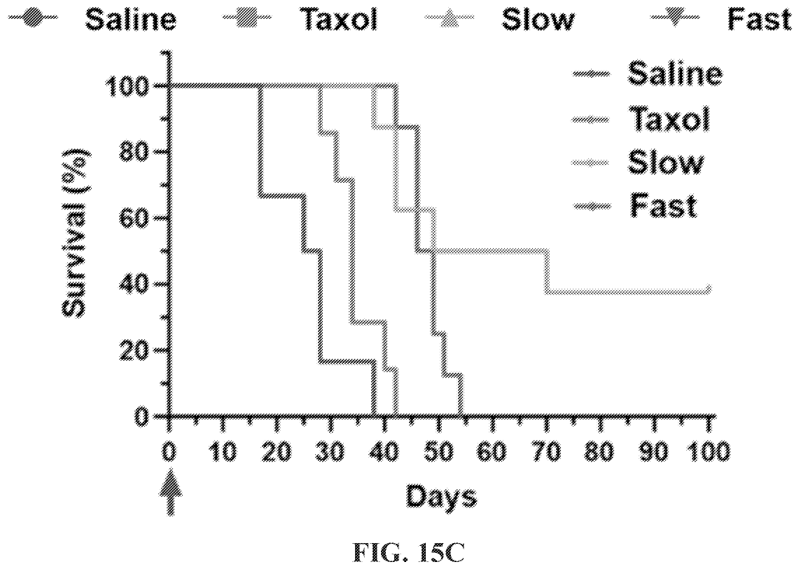

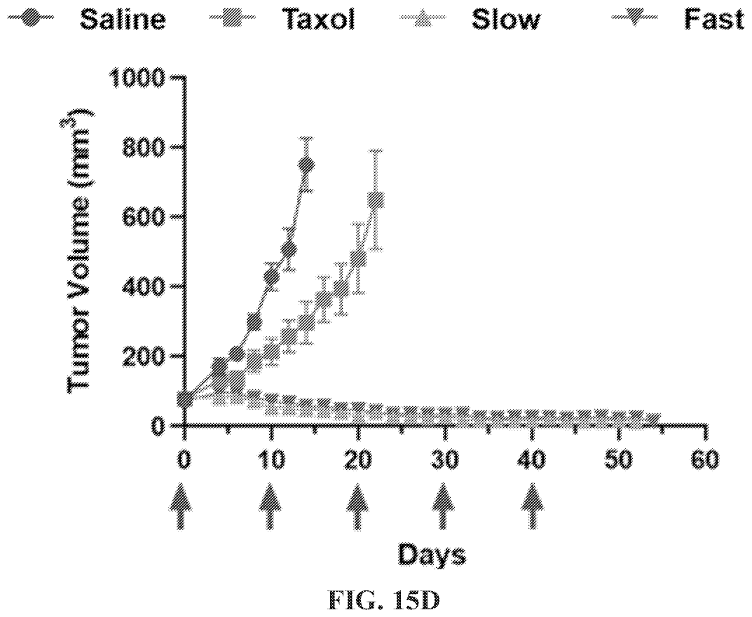

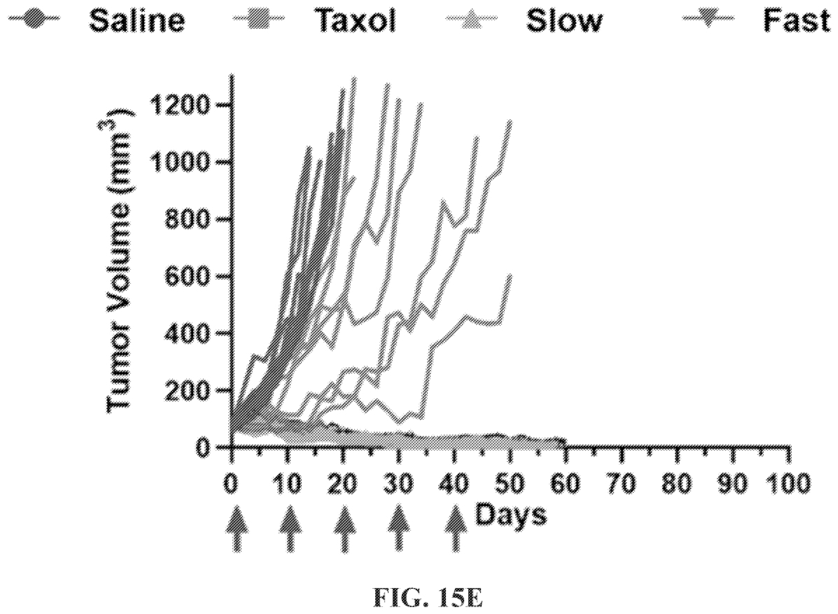

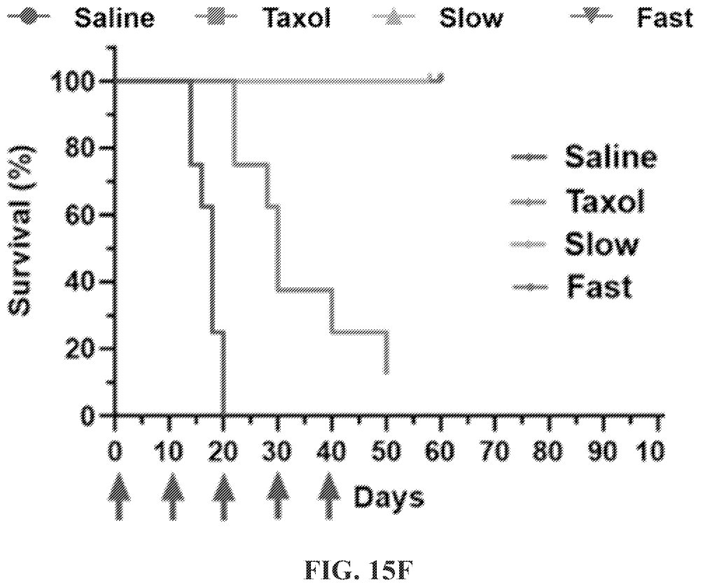

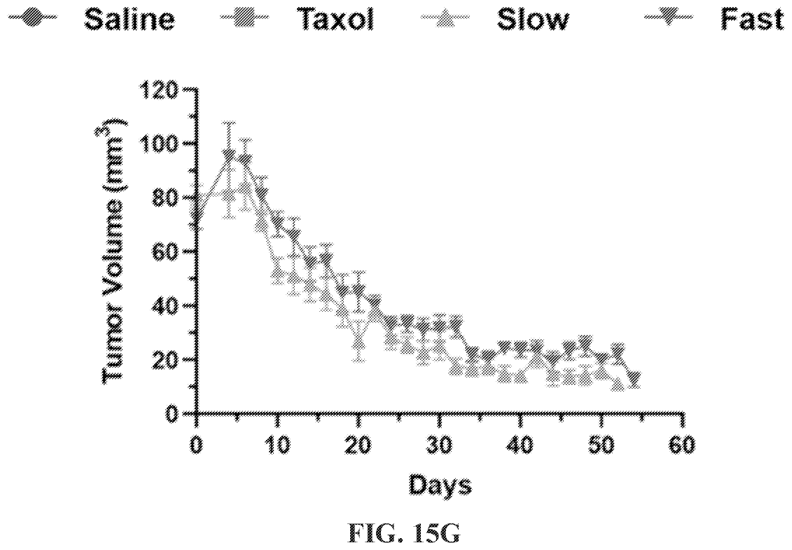

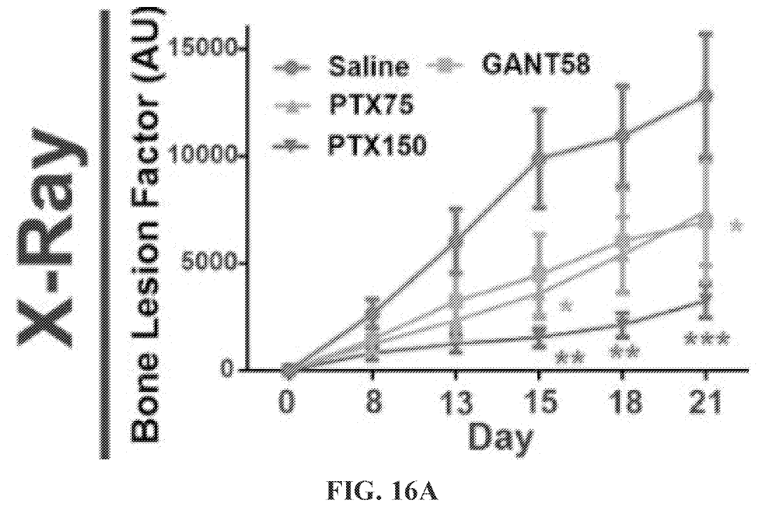

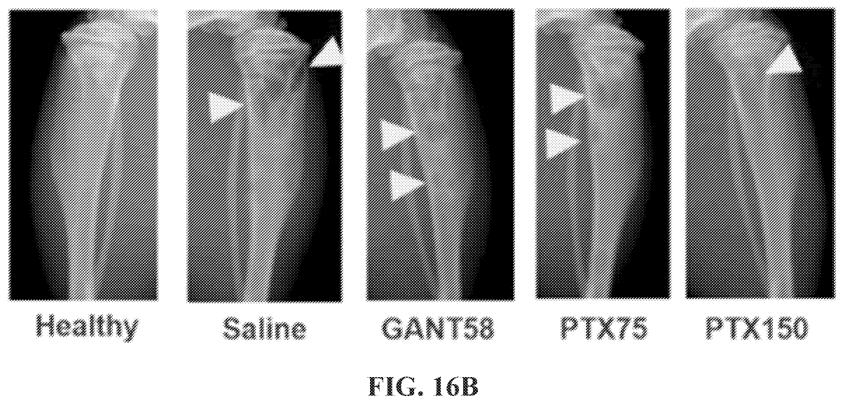

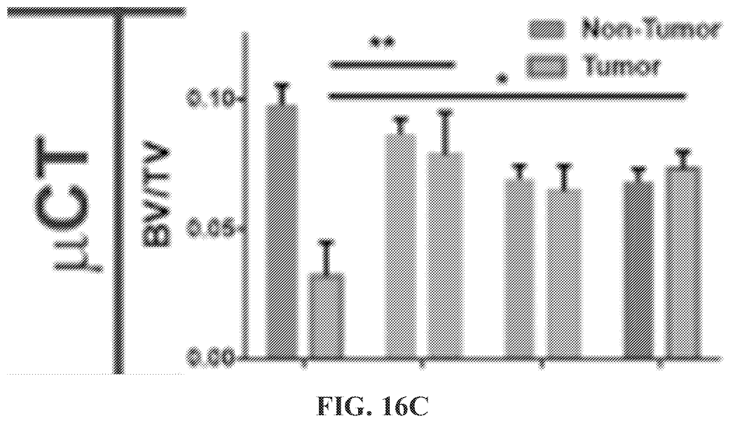

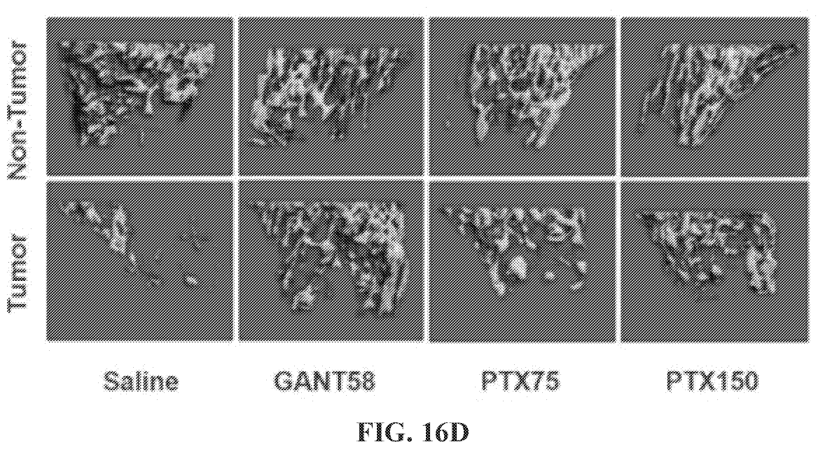

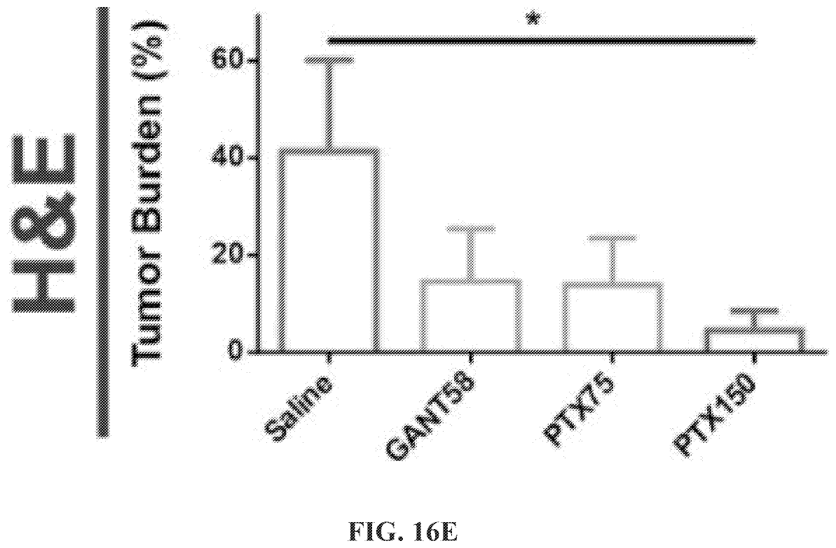

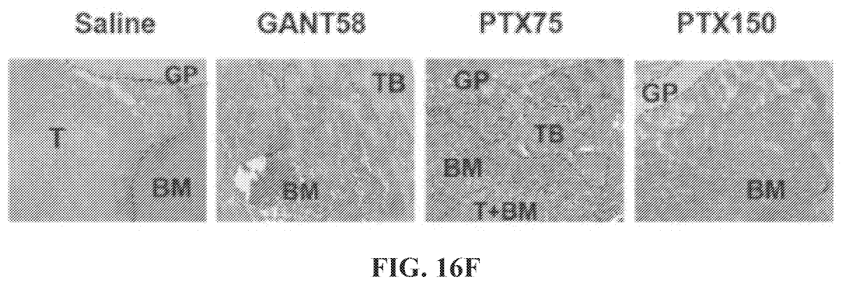

Further, it is reported using the 80% Bz:20% OH formulation and 40% Bz:60% OH formulation, an MTD of paclitaxel of 150 mg/kg in BALB/c mice (FIG. 13A-B) as well as therapeutic outcomes for the same formulations (FIG. 15A-15G). The joint highest MTD reported in the literature and is significantly higher than reported values for clinical formulations: Taxol (20 mg/kg), Genexol PM (50 mg/kg), and Abraxane (90 mg/kg). The poly(oxazolines) based (POx) polymers which also have an MTD of 150 mg/kg are now being commercialized by DelAqua Pharmaceuticals Inc. It should be noted that previous studies have demonstrated that higher doses of drug, permitted through higher MTD values leads to significantly better responses (e.g. in antitumoral activity of paclitaxel loaded nanoparticles). In agreement with these studies, it was found that 150 mg/kg outperformed 75 mg/kg dosing using the present 80% Bz polymer in an aggressive bone metastasis model (4T1 bone clone tumor cells intratibial injected in BALB/.c mice, treated 2× per week) with 150 mg/kg treated mice on average showing complete tumor eradication in 5/8 mice and more than a 50% reduction in tumor volume vs 75 mg/kg treated mice (FIG. 16A-16F). In the same study, GANT58 was also evaluated at 75 mg/kg; previously, GANT58 above 8 mg/kg was unable to be delivered, which required a 5× per week dosing regime and still did not show a reduction in tumor burden. Here, using 75 mg/kg in a 2× per week dosing regime, observed for the first time was a reduction in tumor burden for a GANT58 monotherapy treatment.

Compared with traditional drug-delivery systems, this system has the advantages of 1) tailorability to individual drugs, allowing a large amount of suitable drugs, including traditionally difficult to load peptide-based drugs, to be loaded at high efficiency and capacity, and 2) the polysulfide backbone permits a tailorable ROS-triggered release mechanism. High drug-loading permits the administration of higher doses and thus better treatment outcomes while the triggered release mechanism reduces off-target drug release/side-effect, and allows for tailorability of delivery modality (i.e., if a fast release is required or a slow release is required). For example, in a high frequency dosing regime, a fast release is likely desirable, whereas a slow release may be more desirable in a lower frequency dosing regimen. Furthermore, a strong drug-polymer interaction may lead to low drug leakage in the blood plasma but also may lead to a potentially too slow drug release at target tissue; having a (ROS-)triggered and tunable drug release rate provides various benefits, i.e. high drug-loading, low drug leakage in the blood plasma, and also fast release at the diseased site.

### Example 2

### Ultrahigh Drug-Loaded Micelles Inhibit Bone Destruction and Reduce Tumor Burden in Bone Metastasis

Cancer frequently metastasizes to the bone in breast cancer patients and causes significant bone pain and fracture risk. In bone metastasis, Gli2 transcription factor drives the expression of parathyroid hormone-related protein (PTHrP) resulting in osteoclast maturation and increased bone destruction. Small-molecule inhibitors of Gli2, such as GANT58, are able inhibit tumor-induced bone destruction (TIBD), but promising previous studies have been limited by nanoformulations that have relatively modest wt % drug loading, limiting animal dosing. Herein, a polymeric nanocarrier was developed which permits ultrahigh drug loading through tailored drug-polymer (π-π/H-bond) interactions, thus permitting higher maximum tolerated doses (MTDs). In vivo efficacy of this system was explored for delivery of both GANT58 and paclitaxel (PTX).

Polymeric micelles were tailored using different co-monomer ratios that modulate H-bond and π-π interactions between the polymeric core and drug. Drug loading was achieved using a thin-film rehydration method and loading efficiency/capacity was determined using HPLC (PTX) or fluorescence (GANT58). 6-8-week-old BALB/c female mice were intratibually inoculated with 4T1-cells cloned from a previous bone metastasis and treated 2× per week with saline or ultrahigh GANT58—(75 mg/kg) or PTX—(75 or 150 mg/kg) loaded micelles. Bone X-ray and micro-computed tomography (μCT) were used to measure TIBD, and H&E histological analysis was used to quantify overall tumor burden endpoint analysis (FIG. 16B, FIGS. 16D, and 16F).

Rationally designed polymer micelles were able to load PTX and GANT58 with an LC≥40 wt. % of drug. The MTD of ultrahigh PTX-loaded micelles was determined to be 150 mg/kg in BALB/c mice, the highest reported for a PTX formulation and in agreement with previous findings that indicate higher LCs lead to higher MTDs. In mice intratibially inoculated with 4T1-bone-cloned cells, X-ray and μCT analysis confirmed that all formulations protected bone from TIBD relative to vehicle (saline), with 150 mg/kg PTX providing the strongest bone-protective effects. H&E histological sections confirmed that all of these treatments also significantly reduced tumor burden, again with 150 mg/kg of PTX showing the lowest tumor burden. Complete tumor eradication was observed in 2/7 of (75 mg/kg) GANT58, 4/8 of 75 mg/kg PTX, and 5/8 of 150 mg/kg PTX treated mice.

By tailoring the polymer-drug interaction through co-monomer ratio optimization, polymer formulations capable of ultrahigh drug loading were obtained. Leading formulations effectively prevented TIBD and reduced tumor burden in an aggressive 4T1-bone metastasis murine model.

### Example 3

### Exploring Intermolecular Drug-Nanocarrier Interactions for High Drug Loading

Nanomedicines offer distinct advantages over traditional small-molecular drugs such as improved tropism and reduced side-effects. Despite this, nanomedicines have not lived up to expectations, with relatively few clinical approvals. One contributing factor for this is carrier immunogenicity, which limits the total dose. It has become recently appreciated that higher drug-loading (wt % drug in NP) leads to higher maximum tolerated doses, and improved outcomes. Here, a polymeric nanocarrier library has been developed where the hydrophobic ‘core’ contains varied proportions of H-bonding (monomer A) and π-interacting (monomer B) moieties so as to optimize drug-nanocarrier interactions.

Synthesis: Hydrophobic polymer ‘cores’ were synthesized via anionic ring-opening polymerization of monomer A and monomer B at 100, 90, 80, 60, 50 40 and 30 wt. % of B and end-capped with an alkyne moiety. The core polymers were conjugated to hydrophilic stealth corona block, (poly(dimethylacrylamide) (PDMA)-azide) via the copper-catalyzed Huisgen click-reaction and purified by dialysis.

Drug-loading: Paclitaxel, etoposide, and chloroquine were chosen as they are difficult-to-load drugs containing a mixture of H-bond acceptors/donors and π-interacting groups. Polymer and drug were dissolved at a 1:1 ratio and solvent was removed by nitrogen-flow, followed by high vacuum. Films were rehydrated at 10 mg/mL drug in DI water (stirred at 1200 rpm/50° C.) and then centrifuged for 4 minutes at 7000 rpm; the supernatant was separated from pellet (if any) and loaded drug was determined by HPLC analysis. The resulting concentration was used to calculate loading efficiency (LE) and load capacity (LC); the latter was determined gravimetrically after supernatant lyophilization.

Drugs that have proportionally large potential for both H-bond and π-π interactions were expected to load well into polymeric cores with complimentary interactions. Paclitaxel, which contains both H-bonding and π-interacting moieties, displayed a loading maximum at 90% B (i.e., high π-interactions+low H-bond). In contrast, etoposide displayed significantly higher (>2×) LE+LC in the absence of any H-bonding groups in the core (100% B), whereas chloroquine had an increased loading with increasing H-bonding.

Highest drug loading occurs when the H-bond and π-π interactions between the core polymer and the drug are maximized. ‘Ultra’-high loading (>35% LC) of paclitaxel and etoposide was achieved after core optimization, which was significantly higher than benchmark controls. Crystallinity of the hydrophobic block and corona interactions likely limit further loading in this system for etoposide.

### Example 4

Ultrahigh Drug Loading NPs with ROS-Triggered Release Retain GANT58

Micellar NPs are susceptible to cargo burst release in serum due to dilution and exposure to serum proteins. Premature release increases exposure of free drug, reduces MTD and target site retention, reduces efficacy, and necessitates higher treatment frequency. One of the most promising preclinical micelle chemistries is the polyoxazoline (Pox) class, which achieves a PTX loading capacity (50 wt. %) and drug solubilization concentration (45 g/L). These “ultra-high” loading values are enabled by hydrogen-bonding between the H-bond acceptor amide group of pBuOX and H-bond donor hydroxyls on the drug (e.g., paclitaxel). However, aromatic substitution onto carriers increases drug loading and stability of aromatic-containing drugs such as paclitaxel due to π-π stacking between aromatic groups (of which GANT58 has 5); these interactions are much stronger than hydrophobic interactions conventionally used for loading.

This example describes the development of the first ultrahigh drug loading, ROS-triggered NPs to highly retain GANT58, an entirely aromatic drug with 4H-bond accepting pyridine moieties. These NPs enable ROS-triggered release for 1 week in bone tumors. A polysulfide NP core polymer was rationally designed with Bz (n-stabilization) and OH (H-bond donor and acceptor) side-chains to maximize core-drug stabilizing interactions. An extremely controlled and efficient synthesis approach was also developed for this system, amenable to scale for clinical production. Episulfide (Bz and OH) monomers were copolymerized from a dithiol initiator via an anionic ring-opening polymerization (ROP), analogous to epoxide polymerization (e.g. of ethylene oxide to PEG) but less sensitive to H₂O contamination and yielding polymers with low dispersity (typically, Ð=1.05-1.2). The custom Bz and OH monomers were derived from commercially sourced epoxide monomers through a facile 1-step conversion which does not require chromatographic purification steps. In this instance, 25 g of epoxide was converted to the corresponding episulfide, providing 23 g of product, in a half-day (from start of reaction to obtaining final product). The NP core-forming polymer was chain extended with poly(dimethylacrylamide) (PDMA) via Reversible Addition-Fragmentation Chain-Transfer (RAFT) polymerization to yield a triblock copolymer with Bz-sulfide-co-OH— sulfide random copolymer core (PDMA₄ₖ-b-PTE(Bzₓ-ran-OH_{y})₃ₖ-b-PDMA₄ₖ). As with ROP, RAFT produces low dispersity polymers and is scalable to kg-sized batches.

PDMA₄ₖ-b-PTE(Bzₓ-ran-OH_{y})₃ₖ-b-PDMA₄ₖ was successfully synthesized with a benzyl (B) core wt. % of 100, 90, 80, 70, 60, 50 and 40% as confirmed by ¹H NMR and GPC analysis; the balance was the OH functional sulfide monomer (0, 10, 20, 30, 40, 50, 60 wt. %). Drug loading feed was set at a polymer:GANT58 ratio of 1:1 using a thin-film rehydration method similar to that described for POx-polymers and Genexol. High LCs (wt/c drug in final NP) of ˜40-50% and LEs of ˜35-45% (% drug in feed loaded into NP; yields 3.5-4.5 g/L GANT58) were obtained for the 50-100% B formulations. This was significantly higher than the benchmarks of Pluronic F127 (9/9%), PEG-PLGA (5/4%), corresponding PPS core triblocks (24/21%), or 2^{nd} generation PPS diblock micelles (dashed line, 16/30%). Micelles (at 0.1 mg/mL) with a B wt. % of ≥60% were found to be highly serum stable (≤10% release of FRET pair DiO/DiI after 24 hrs in 50% serum).

The polysulfide backbones have a unique oxidation-responsive mechanism, by which oxidation converts the polymers to a more hydrophilic sulfide/sulphone and triggers release at tumors and other sites characterized by high oxidative stress. It was observed that increasing wt. % of the hydrophilic OH monomer relative to the Bz monomer accelerated oxidation-triggered release due to the increasing solubility of oxidant (H₂O₂) in the polysulfide core. This permitted tailoring the drug release kinetics at the tumor site for optimal therapeutic efficacy across a set of micelles that have similar drug loading capacity (60-100% B).

### Example 5

### Materials and Methods for Examples 1-4

Monomer Synthesis of Thioglycidyl Benzyl Ether (BzTG). 25.0 g of glycidyl benzyl ether (152.25 mmol), 23.2 g of thiourea (304.5 mmol, 2 eq.s), and 8.1 g of NH4Cl were introduced into a reaction flask with 250 mL of MeOH. The reaction flask was transferred to a heating bath set at 60° C. and the reaction was stirred under a nitrogen atmosphere for 1.5 hours or until the starting material had been fully consumed (followed by TLC—a significant amount of side products form if allowed to react for too long). Volatiles were removed by rotary evaporation and the multiphasic residues were dissolved into a biphasic mixture of diethyl ether and water, transferred to a separating funnel and 250 mL of ether was extracted against 50 mL of water three times, followed by brine two times. The organic phase was collected, dried over Na₂SO₄, filtered, and concentrated in vacuo. The faintly yellow oil was then purified through a short column of silica using hexane as the eluent. The hexane was removed by rotary evaporation to give 22.3 g of a colorless oil product (81.3% yield).

Monomer Synthesis of Tert-Butyldimethylsilyl Thioglycidyl Ether (TBTE). Tert-butyldimethylsilyl thioglycidyl ether (TBTE) was synthesized using an identical procedure as thioglycidyl benzyl ether (BzTG). Briefly, 25.0 g of tert-butyldimethylsilyl glycidyl ether (132.74 mmol), 20.21 g of thiourea (265.48 mmol, 2 eq.s), and 8.1 g of NH₄Cl were introduced into a reaction flask with 250 mL of MeOH. The reaction flask was transferred to a heating bath set at 60° C. and the reaction was stirred under a nitrogen atmosphere for 1.5 hours or until the starting material had been fully consumed (followed by TLC—a significant amount of side products form if allowed to react for too long). Volatiles were removed by rotary evaporation and the multiphasic residues were dissolved into a biphasic mixture of diethyl ether and water, transferred to a separating funnel and 250 mL of ether was extracted against 50 mL of water three times, followed by brine two times. The organic phase was collected, dried over Na₂SO₄, filtered, and concentrated in vacuo. The faintly yellow oil was then purified through a short column of silica using hexane as the eluent. The hexane was removed by rotary evaporation to give 20.8 g of a colorless oil product (76.7% yield).

Synthesis of Difunctional Initiator. 10 g (54.85 mmol) of 2,2′-(Ethylenedioxy)diethanethiol and 45 mL of triethylamine (329.12 mmol, 6 equiv.s) were introduced into 400 mL of dried THF under an argon atmosphere. The reaction flask was transferred to an ice bath and a chilled solution of 19.57 mL of acetyl chloride (165.6 mmol, 5 equiv.s) in 50 mL of dry THF was added dropwise under vigorous stirring. The mixture was removed from the ice bath and allowed to react at room temperature for an additional 4 hr, then filtered, and volatiles were removed by rotary evaporation. The resulting oil was diluted with 150 mL of dichloromethane, washed with brine (3×), dried over Na₂SO₄, filtered, and concentrated by rotary evaporation. The residue was further purified by column chromatography using hexane:ethyl acetate (3:1) as the mobile phase and silica gel as the stationary phase to give 11.23 g of a colorless oil (76.9% yield).

Polymer Synthesis of Poly(BzTG-co-TBTE) Dialkyn. Polysulfides were synthesized via an anionic ring-opening polymerization. For 1 gram of polymer/monomer, 1 eq. of DODTA was added to 5 mL of degassed THF with 6 eq.s of tributylphosphine under a nitrogen atmosphere. 2.1 eq.s of sodium methoxide was then added as a 0.5 M methanolic solution, followed 5 minutes later by an appropriate amount of BzTG and TBTE; 16:0, 15:3, 11.7:10, 10:13, 8.3:16.7, 6.1:20, 5:23.3 (BzTG:TBTE) respectively for the 100, 90, 80, 70, 60, 50, 40 and 30 wt. % BzTG (of final deprotected polysulfide). The reactants were allowed to react for 5 hours and quenched by the addition of 5 eq.s of propargyl bromide, which was then further allowed to react for another 1 hour. The solution was diluted with 40 mL of dichloromethane and extracted against 5 mL of brine 3×, dried over Na₂SO₄, filtered, and concentrated in vacuo. The oil was dissolved in a minimum amount of DCM and precipitated into 5 mL of methanol and then centrifuged. Supernatant was removed and the oil was washed with 4 mL of fresh methanol. The resulting oil was dried in a vacuum oven for 48 hours to give the final product.

Deprotection of Alcohol. For 1 mass of polysulfide, 1 mass of DOWEX 1×8 acid resin was weighted. DOWEX was first washed 2× with tetrahydrofuran (THF), 2× with methanol, followed by activation with 1 M HCl (2×). The solution was aspirated and the polysulfide dissolved in THF:methanol (8:2) was added to the DOWEX resin and allowed to react for 24 hours at 45° C. (no stirring). Additional methanol was added to bring the mixture to a 1:1 THF:methanol mixture, which was further reacted for 24 hours. The mixture was concentrated in vacuo and precipitated into cold methanol. The solvent was decanted, and the precipitate was placed in a vacuum oven for 48 hours to yield a clear colorless viscous oil.

Polymer Synthesis of Polypropylene sulfide (PPS) Dialykne. PPS-dialykne was synthesized in an analogous procedure as poly(BzTG-co-TBTE) dialkyne. Briefly, 89.8 mg of DODTA (0.33 mmol) was added to 5 mL of degassed THF with 0.5 mL of tributylphosphine (2.02 mmol, 6 eq.s) under a nitrogen atmosphere. 1.42 mL of sodium methoxide (0.71 mmol, 2.1 eq.s) was then added as a 0.5 M methanolic solution, followed by 1.0 g of propylene sulfide (13.49 mmol, 40 eq.s) 5 minutes later. Reactants were allowed to react for 3 hours under a nitrogen atmosphere and the reaction was quenched with 200.5 mg of propargyl bromide (1.69 mmol, 5 eq.s) for 1 hour. The solution was diluted into 40 mL of dichloromethane and extracted against 5 mL of brine 3×, dried over Na₂SO₄, filtered, and concentrated in vacuo. The oil was dissolved in a minimum amount of DCM and precipitated into 5 mL of methanol and then centrifuged. Supernatant was removed and the oil was washed with 4 mL of fresh methanol two more times. The resulting oil was dried in a vacuum oven for 48 hours to give the final product as a clear, colorless oil.

Synthesis of 4-cyano-4-(ethylsulfanylthiocarbonyl) sulfanylpentanoic acid (ECT). To a 100 mL solution of diethyl ether containing 5.0 g of ethanethiol (80.5 mmol) and 2.01 g of sodium hydride (83.7 mmol, 1.04 eq.s), 5.05 mL of carbon disulfide (83.7 mmol, 1.04 eq.s) was added. The solution was allowed to react for 1 hour and the resulting sodium S-ethyl trithiocarbonate was further reacted with 6.74 g of iodine (26.6 mmol, 0.33 eq.s) to obtain bis(ethylsulfanythiocarbonyl) disulfide. This yellow solid was separated from the ether solution, dissolved into ethyl acetate, and refluxed with 3.38 g of 4,4′-azobis(4-cyanopentanoic acid) (120.7 mmol, 1.5 eq.s) for 24 hr. The volatiles were removed via rotary evaporation and the crude ECT was purified by silica column chromatography using a 1:1 mobile phase of ethyl acetate and hexane. After evaporation of the mobile phase in vacuo, 15.983 g of an orange oil was formed that later formed a yellow powder after storage at −20° C. (75.2% yield).

Synthesis of 3-Azido-propylamine. 15.0 g of 3-bromopropylamine hydrobromide (68.52 mmol) and 17.8 g of sodium azide (274.1 mmol, 4 eq.s) were dissolved into 200 mL of deionized water and magnetically stirred at 80° C. for 24 hours. The solution was allowed to cool to room temperature and was then placed in an ice bath, basified with 10.964 g of sodium hydroxide, saturated with sodium chloride, and extracted against 75 mL of dichloromethane five times. The organic phases were collected, dried over anhydrous potassium carbonate, filtered, and concentrated in vacuo. 5.12 g of product was collected as a 40 wt. % solution in dichloromethane (74.5% yield).

Synthesis of Azido-ECT RAFT Chain Transfer Agent (N3-ECT). To an ice bath-cooled solution containing 6.0 g of ECT (22.78 mmol) in 100 mL of dry ACN, 2.28 g of azido-propyl-amine (22.78 mmol, 1 eq.) and 278 mg of DMAP (1.139 mmol, 0.1 eq.s) in 10 mL of dry ACN was added, followed immediately by 4.8 g of EDC (25.06 mmol, 1.1 eq.s). The reaction flask was removed from the ice-bath and allowed to react at room temperature overnight. The reaction solvent was evaporated in vacuo and redissolved into 150 mL of ethyl acetate and extracted against 35 mL of brine three times. The organic fraction was collected, dried over MgSO₄, filtered, and concentrated by rotary evaporation. The resulting oil was purified by column chromatography using silica as a stationary phase and a 98:2 mixture of dichloromethane:methanol as the mobile phase. This yielded 5.651 g of a dark orange viscous oil (71.8% yield).

Synthesis of N3-Poly(Dinethylacrylamide)(PDMA). 982 mg of N3-ECT (2.52 mmol), 10.0 g of dimethylacrylamide (100.9 mmol, 40 eq.s), and 122 mg of VA-044 (0.378 mmol, 0.15 eq.s) pre-dissolved in a minimum amount of water was added to 30 mL of methanol. The mixture was purged with nitrogen for 30 minutes, sealed, heated to 55° C., and allowed to react overnight. PDMA was purified by precipitation into diethyl ether from dichloromethane three times. The resulting viscous oil was placed in a vacuum oven overnight and yielded a solid glassy yellow product.

Synthesis of PDMA-Polysulfide-PDMA Triblock Copolymer. 45 mg of a ˜3 kDa polysulfide-dialykne (0.015 mmol) was dissolved in 4 mL of dimethylformamide (DMF) together with 180 mg of a ˜4 kDa N3-PDMA (0.045 mmol, 3 eq.s [1.5 eq.s per alkyne]) and 9.6 mg of CuSO₄·5H₂O (0.06 mmol, 4 eq.s). The solution was purged with nitrogen gas for 10 minutes, then under a nitrogen flow, 16 μL of N″,N″-pentamethyldiethylenetriamine (PMDETA) (0.075 mmol, 5 eq.s) and 23 mg of sodium ascorbate (0.12 mmol, 8 eq.s) was added to the reaction mixture, which was then magnetically stirred for 2 days under a nitrogen atmosphere. The reaction mixture was exposed to air and copper was allowed to oxidize overnight. The following day, the mixture was saturated with ethylenediaminetetraacetic acid tetrasodium salt dihydrate (EDTA) and stirred for at least 6 hours. The mixture was diluted with 20 mL of dialysis buffer containing 0.1% EDTA and 10 mM of Na₂HPO₄ buffered to pH 7.5 with 1 M aqueous NaOH or HCl and placed into a dialysis bag with a 12-14 kDa molecular weight cut off (MWCO) pore size. The mixture was dialyzed in 4 L of dialysis buffer for 3 days, with the buffer changed every 2 hours during the day, then a further 2 days in deionized water. The resulting mixture was concentrated in a centrifugal spin filter (Amicon® Ultra-15 Centrifugal Filter Unit) with a MWCO of 10 kDa and 5 further volumes of deionized water was passed through the sample. The resulting solution was frozen and placed in a lyophilizer for 3 days, resulting in a white foamy powder.

Critical Micelle Concentration (CMC). In a black 96-well plate, 200 μL of phosphate buffered saline (PBS) containing dispersions of PDMA-Polysulfide-PDMA triblock copolymers at 0.75, 0.5, 0.4, 0.25, 0.01, 0.075, 0.05, 0.025, 0.01, 0.0075, 0.005, and 0.0001 mg/mL were prepared. To each well, 10 μL of a 0.1 mg/mL solution of Nile Red in acetone was added. The plate was covered in foil and acetone was allowed to evaporate overnight at room temperature. Nile Red fluorescence was measured using an excitation wavelength of 540±20 nm and an emission wavelength of 620±20 nm. Each condition was performed in triplicate and the CMC was defined as the inflection point between linear fittings of the average of low concentrations and the average of linear fittings of high concentrations. A summary of CMC and DLS sizing parameters is shown below in Table 2.

TABLE 2
Summary of CMC and DLS Sizing Parameters.

| | | Unloaded | | PTX-Loaded | | | |
|---|---|---|---|---|---|---|---|
| | CMC | Z- | | 7- | | LE | LC |
| Core | (mg/mL) | Ave | PDI | Ave | PDI | (%) | (%) |
| P(BzTG₁₆) | 0.060 | 306.8 | 0.554 | | | 40.8 | 30.4 |
| P(BzTG₁₅-TGG₃) | 0.061 | 157.2 | 0.549 | | | 72.3 | 45.3 |
| P(BzTG_{13.3}-TGG_{6.7}) | 0.055 | 104.1 | 0.429 | | | 63.2 | 38.0 |
| P(BzTG_{11.7}-TGG₁₀) | 0.062 | 81.03 | 0.461 | | | 65.0 | 38.3 |
| P(BzTG₁₀-TGG_{13.3}) | 0.059 | 61.24 | 0.63 | | | 46.9 | 32.0 |
| P(BzTG_{8.3}-TGG_{16.7}) | 0.061 | 52.36 | 0.715 | | | 39.4 | 35.0 |
| P(BzTG_{6.7}-TGG₂₀) | 0.065 | 68.88 | 0.814 | 212.8 | 0.224 | 37.5 | 34.2 |
| P(BzTG₅-TGG_{23.3}) | 0.045 | 64.34 | 0.77 | 218.9 | 0.137 | 30.3 | 27.0 |
| PPS | 0.058 | 294 | 0.485 | | | 23.8 | 27.3 |
| Pluronic-F127 | ~0.869 | | | | | 29.5 | 18.5 |
| PEG₅ₖ-b-PLGA₁₀ₖ | — | | | | | 6.0 | 9.0 |
| PEG₂ₖ-b-PLA₂ₖ | 0.0611 | | | | | 13.6 | 13.4 |

Serum Stability Experiment. Serum stability was determined using the Forster Resonance Energy Transfer (FRET) pair DiO and DiI in a similar manner as previously described (Vanderburgh et al., ACS Nano. 2020, 14, 1, 311-327, which is incorporated by reference herein in its entirety). Briefly, triblock PDMA-polysulfide-PDMA polymers were dissolved in chloroform or a chloroform:methanol mixture with 2 mg of DiI and 2 mg of DiO and evaporated to dryness to form a thin film. This thin film was protected from light and rehydrated in 0.4 mL of deionized water for 25 minutes under a vigorous stir (1100 rpm) at 50° C. The dispersion was then transferred into a 2 mL Eppendorf tube and centrifuged at 8000×g for 5 minutes in a ThermoScientific Sorvall Legend Micro 21r centrifuge to remove unloaded fluorophore. For all polymers, no pellet was detected. The fluorophore-loaded polymers were diluted to 0.1 mg/mL (of polymer) in 50% FBS serum (fetal bovine serum in PBS) and immediately added to a 96-well plate and sealed with a transparent film. In a plate reader, the fluorescence was measured at 517 nm and 573 nm using an excitation wavelength of 480 nm over 24 hours at 37° C. The FRET efficiency was determined using the following equation:

$$\% FRET = [ {I}_{573} \div ( {I}_{573} + {I}_{517} ) ] \times 100.$$

Oxidation Experiments. To each well of a 96-well plate, 190 μL of a 0.25 mg/mL aqueous solution of PDMA-b-Polysulfide-b-PDMA triblock copolymers was added, followed by 5 μL of a 0.2 mg/mL solution of Nile Red in acetone. Acetone was allowed to evaporate over 4 hours at room temperature. 10 μL of a 10 M H₂O₂ solution was then added to each well, sealed with transparent film, and Nile Red fluorescence (ex. 540 nm, em. 620 nm) was read at intermittent time intervals over 48 hours. A control group without H₂O₂ addition was used to determine the bleaching of Nile Red overtime and fluorescence was normalized as previously described (El Mohtadi et al., Biomacromolecules. 2020, 21, 2, 305-318, which is incorporated by reference herein in its entirety) using the following equation:

[emission intensity at a given time point]÷[emission intensity at time=0×the fraction of unbleached dye].

Each reading was the average of 3 replicates.

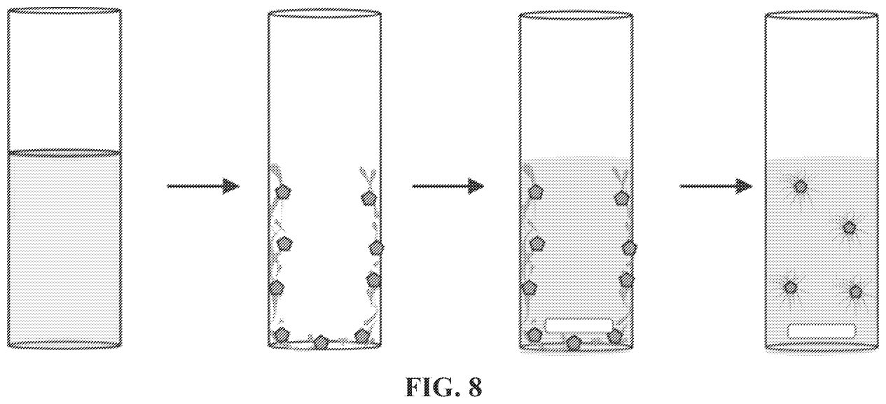

Drug Loading Experiments. 4 mg of drug and 4 mg of polymer were dissolved into 0.3 mL of chloroform or a chloroform-methanol solvent mixture, transferred to a 4 mL glass vial, and evaporated to dryness to produce a drug-polymer thin film. 0.4 mL of deionized water was added to the vial, sealed, and vigorously stirred at 1100 rpm at 50° C. for 25 minutes (FIG. 8). The dispersion was then transferred into a 2 mL Eppendorf tube, placed in a sonication bath, and sonicated for 15 minutes. The tube was then centrifuged at 8000×g for 5 minutes in a ThermoScientific Sorvall Legend Micro 21r centrifuge and supernatant was separated from any pellets. Loading was determined using fluorescence, if possible, or high performance liquid chromatography (HPLC) using a C18 column.

### Example 6

### Example Polymers of Second Block

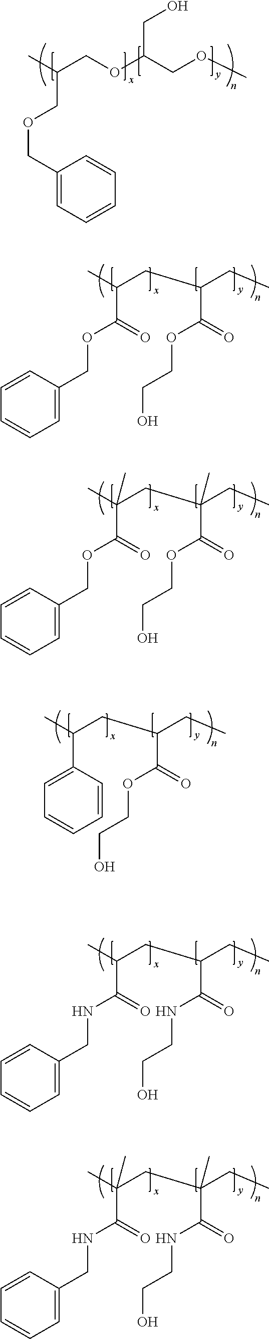

Top left polymer: The polyglycidyl polymers synthesized via for example anionic or cationic ring-opening copolymerization of oxirane/epoxide/glycidyl polymer.

Remaining polymers: Copolymers can be synthesized via either controlled radical polymerization such as reversible addition fragmentation chain transfer (RAFT), atom transfer radical polymerization (ATPR), stable free radical mediated polymerization (e.g. nitroxide mediated polymerization or verdazyl mediated polymerization) or iniferter polymerization or (uncontrolled) free radical polymerization. Monomers could include acrylates (Top middle), methacrylates (Top right), styrenes (bottom left), acrylamides (bottom middle), methacrylamides (bottom right) as well as any other vinylic monomer such as vinyl acetates and vinyl acetamides or any copolymer mixture of these stated monomers.

Alternatives to anionic ring-opening polymerization of thiiranes include cationic ring-opening polymerization, thioacyl group transfer and decarboxylative ROP of cyclic thiocarbonates or epoxide+carbonyl sulfide (COS) comonomer mixtures (see, e.g., Zhang et al. Poly(thioether)s from Closed-System One-Pot Reaction of Carbonyl Sulfide and Epoxides by Organic Bases, J. Am. Chem. Soc. 2019, 141, 13, 5490-5496, which is incorporated by reference herein in its entirety).

Thietanes also undergo ring-opening polymerizations and can be an alternative monomer to thiiranes (also known as episulfides) that also produce polysulfides.

It is understood that the foregoing detailed description and accompanying examples are merely illustrative and are not to be taken as limitations upon the scope of the invention.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, the following Embodiments are provided.

Clause 1. A block copolymer comprising: a first block (A), wherein the first block comprises a hydrophilic polymer; and a second block (B), wherein the second block comprises recurring units of formula (I)

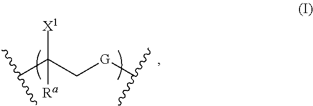

wherein:
X¹ is of formula (II)

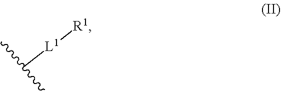

or
formula (III)

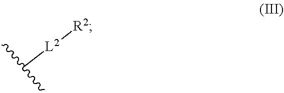

L¹ and L² are each independently —C(O)O-alkylene, —C(O)NR^{b}-alkylene, alkylene, alkylene-O-alkylene, alkylene-O—, or bond; R¹ is a π-interacting moiety: R² is a hydrogen donating moiety; R¹ is hydrogen or C₁₋₃alkyl; R^{b} is hydrogen or C₁₋₆alkyl; and G is S, O, or bond.

Clause 2. The block copolymer of clause 1, wherein: R¹ is an aryl or heteroaryl, wherein the aryl and heteroaryl are optionally substituted with 1 or 2 substituents, each independently halogen, cyano, C₁₋₄alkyl, C₁₋₂fluoroalkyl, —OC₁₋₂alkyl, or —OC₁₋₂fluoroalkyl; and R² is hydroxy, amine, or amide.

Clause 3. The block copolymer of clause 1 or 2, wherein: L¹ and L² are each independently —C(O)O—C₁₋₄alkylene, —C(O)NH—C₁₋₄alkylene, C₁₋₁₀alkylene, C₁₋₆alkylene-O—C₁₋₆alkylene, C₁₋₄alkylene-O—, or bond.

Clause 4. The block copolymer of any one of clauses 1-3, wherein: L¹ and L² are each independently C₁₋₁₀alkylene or C₁₋₆alkylene-O—C₁₋₆alkylene; R¹ is aryl or heteroaryl; R² is hydroxy or amine; and G is S or O.

Clause 5. The block copolymer of any one of clauses 1-4, wherein: X¹ is of formula (II-a)

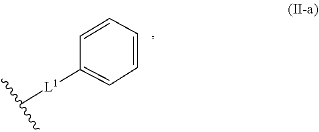

or
formula (III-a)

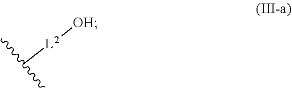

L¹ is C₁₋₄alkylene or C₁₋₂alkylene-O—C₁₋₂alkylene; L² is C₁₋₄alkylene; R^{a} is hydrogen; and G is S.

Clause 6. The block copolymer of any one of clauses 1-5, wherein the recurring unit of formula (I) is repeated 5 to 350 times.

Clause 7. The block copolymer of any one of clauses 1-6, wherein the second block comprises recurring units including formula (II) and recurring units including formula (III) at a weight ratio of about 100:0 to about 30:70 (formula (II):formula (III)).

Clause 8. The block copolymer of any one of clauses 1-7, wherein the second block comprises recurring units including formula (11) at about 30% to about 100% by weight of the second block.

Clause 9. The block copolymer of any one of clauses 1-8, wherein the hydrophilic polymer comprises poly(dimethylacrylamide)(PDMA), poly(ethylene glycol) (PEG), poly(PEG), poly(methyl oxazoline)(PMOX), poly(ethyl oxazoline), polysarcosine, poly(4-acryloylmorpholine), poly(glycerol monomethacrylate), poly(propylene sulfoxide), poly(2-(methylsulfinyl)ethyl acrylate)(PMSEA), poly(vinyl alcohol)(PVA), poly(glycidol), poly(thioglycidyl glycerol)(PTGG), poly(2-methacryloyloxyethyl phosphorylcholine)(PMPC), poly(vinyl pyrrolidone)(PVP), poly(N-(2-hydroxypropyl)methacrylamide)(PHPMA), poly(trimethylamine N-oxide), poly(lysine-methacrylamide), poly(lysine-acrylamide), poly(carboxybetaine), poly(sulfobetaine), Heparosan, or a combination thereof.

Clause 10. The block copolymer of any one of clauses 1-9, wherein the first block has a number average molecular weight of about 0.5 kDa to about 40 kDa.

Clause 11. The block copolymer of any one of clauses 1-10, wherein the second block has a number average molecular weight of about 1 kDa to about 50 kDa.

Clause 12. The block copolymer of any one of clauses 1-11, wherein the block copolymer is an A-B diblock copolymer, an A-B-A triblock copolymer, a B-A-B triblock copolymer, or a star multiblock copolymer.

Clause 13. The block copolymer of any one of clauses 1-12, wherein the block copolymer has a number average molecular weight of about 2 kDa to about 100 kDa.

Clause 14. The block copolymer of any one of clauses 1-13, wherein the block copolymer is A_{0.5 kDa-40 kDa}-B_{1 kDa-50 kDa} or A_{0.5 kDa-40 kDa}-B_{1 kDa-50 kDa}-A_{0.5 kDa-40 kDa}.

Clause 15. The block copolymer of any one of clauses 1-14, wherein: the hydrophilic polymer comprises PDMA, PEG, poly(glycerol monomethacrylate), or a combination thereof; and the second block includes recurring units of formula (I-a)

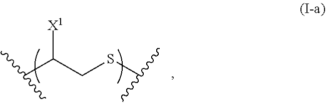

wherein: X¹ is of formula (II-a)

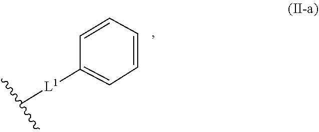

or
formula (III-a)

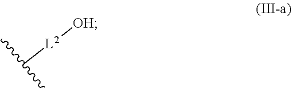

L¹ is C₁₋₄alkylene or C₁₋₂alkylene-O—C₁₋₂alkylene; and L² is C₁₋₄alkylene.

Clause 16. A composition comprising: a plurality of block copolymers according to any one of clauses 1-15 self-assembled into a particle; and a drug encapsulated within the particle.

Clause 17. The composition of clause 16, wherein the drug comprises a hydrogen bond acceptor moiety, an aromatic moiety, or a combination thereof.

Clause 18. The composition of clause 16 or 17, wherein the drug comprises a peptide-based drug, a chemotherapeutic, an anti-inflammatory drug, an immune modulating drug, or a combination thereof.

Clause 19. The composition of any one of clauses 16-18, wherein the drug comprises cyclosporin A, paclitaxel, bortezomib, etoposide, neratinib, osimertinib, chloroquine, GANT58, docetaxel, dexamethasone, carmofur, dexamethasone, dexamethasone palmitate, carfilzomib, afatinib, irinotecan, doxorubicin, doxycycline, camptothecin, imiquimod, MK-8617, ciclopirox, roxadustat, or a combination thereof.

Clause 20. The composition of any one of clauses 16-19, wherein the drug has a log D of about −1 to about 10.

Clause 21. The composition of any one of clauses 16-20, wherein the particle comprises the drug at about 0.5% to about 60% by weight of the particle.

Clause 22. The composition of any one of clauses 16-21, wherein the particle has a diameter of about 10 nm to about 500 nm.

Clause 23. A method of treating a disease or disorder in a subject in need thereof, the method comprising administering to the subject a therapeutically effective amount of the composition of any one of clauses 16-22.

Clause 24. The method of clause 23, wherein the disease or disorder is cancer, osteoarthritis, rheumatoid arthritis, ulcerative colitis, or wound healing.

Clause 25. The method of clause 23 or 24, wherein the composition has a maximum tolerated dose of at least 150 mg/kg.

What is claimed is:

1. A block copolymer comprising:
a first block (A), wherein the first block comprises a hydrophilic polymer; and
a second block (B), wherein the second block comprises recurring units of formula (I) 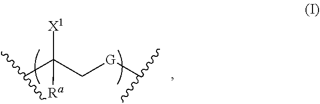
wherein:
X¹ is of formula (II) 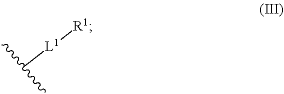
or
formula (III) 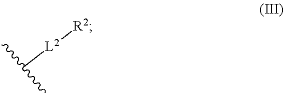
L¹ and L² are each independently —C(O)O-alkylene, —C(O)NR^{b}-alkylene, alkylene, alkylene-O-alkylene, alkylene-O—, or bond;
R¹ is a π-interacting moiety;
R² is a hydrogen donating moiety;
R^{a} is hydrogen or C₁₋₃alkyl;
R^{b} is hydrogen or C₁₋₆alkyl; and
G is S, O, or bond.

2. The block copolymer of claim 1, wherein:
R¹ is an aryl or heteroaryl, wherein the aryl and heteroaryl are optionally substituted with 1 or 2 substituents, each independently halogen, cyano, C₁₋₄alkyl, C₁₋₂fluoroalkyl, —C₁₋₂alkyl, or —OC₁₋₂fluoroalkyl; and
R² is hydroxy, amine, or amide.

3. The block copolymer of claim 1, wherein:
L¹ and L² are each independently —C(O)O—C₁₋₄alkylene, —C(O)NH—C₁₋₄alkylene, C₁₋₁₀alkylene, C₁₋₆alkylene-O—C₁₋₆alkylene, C₁₋₄alkylene-O—, or bond.

4. The block copolymer of claim 1, wherein:
L¹ and L² are each independently C₁₋₁₀alkylene or C₁₋₆alkylene-O—C₁₋₆alkylene;
R¹ is aryl or heteroaryl;
R² is hydroxy or amine; and
G is S or O.

5. The block copolymer of claim 1, wherein:
X¹ is of formula (II-a) 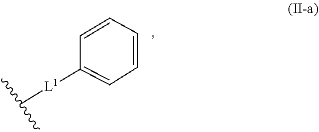
or
formula (III-a) 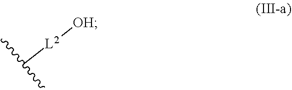
L¹ is C₁₋₄alkylene or C₁₋₂alkylene-O—C₁₋₂alkylene;
L is C₁₋₄alkylene;
R^{a} is hydrogen; and
G is S.

6. The block copolymer of claim 1, wherein the recurring unit of formula (I) is repeated 5 to 350 times.

7. The block copolymer of claim 1, wherein the second block comprises recurring units including formula (II) and recurring units including formula (III) at a weight ratio of about 100:0 to about 30:70 (formula (II):formula (III)).

8. The block copolymer of claim 1, wherein the second block comprises recurring units including formula (II) at about 30% to about 100% by weight of the second block.

9. The block copolymer of claim 1, wherein the hydrophilic polymer comprises poly(dimethylacrylamide)(PDMA), poly(ethylene glycol) (PEG), poly(PEG), poly(methyl oxazoline)(PMOX), poly(ethyl oxazoline), polysarcosine, poly(4-acryloylmorpholine), poly(glycerol monomethacrylate), poly(propylene sulfoxide), poly(2-(methylsulfinyl)ethyl acrylate)(PMSEA), poly(vinyl alcohol)(PVA), poly(glycidol), poly(thioglycidyl glycerol)(PTGG), poly(2-methacryloyloxyethyl phosphorylcholine)(PMPC), poly(vinyl pyrrolidone)(PVP), poly(N-(2-hydroxypropyl)methacrylamide)(PHPMA), poly(trimethylamine N-oxide), poly(lysine-methacrylamide), poly(lysine-acrylamide), poly(carboxybetaine), poly(sulfobetaine), Heparosan, or a combination thereof.

10. The block copolymer of claim 1, wherein the first block has a number average molecular weight of about 0.5 kDa to about 40 kDa.

11. The block copolymer of claim 1, wherein the second block has a number average molecular weight of about 1 kDa to about 50 kDa.

12. The block copolymer of claim 1, wherein the block copolymer is an A-B diblock copolymer, an A-B-A triblock copolymer, a B-A-B triblock copolymer, or a star multiblock copolymer.

13. The block copolymer of claim 1, wherein the block copolymer has a number average molecular weight of about 2 kDa to about 100 kDa.

14. The block copolymer of claim 1, wherein the block copolymer is A_{0.5 kDa-40 kDa}-B_{1 kDa-50 kDa} or A_{0.5 kDa-40 kDa}-B_{1 kDa-50 kDa}-A_{0.5 kDa-40 kDa}.

15. The block copolymer of claim 1, wherein:
the hydrophilic polymer comprises PDMA, PEG, poly(glycerol monomethacrylate), or a combination thereof; and
the second block comprises recurring units of formula (I-a) 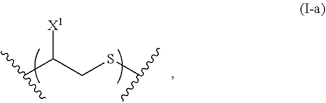
wherein: X¹ is of formula (II-a) 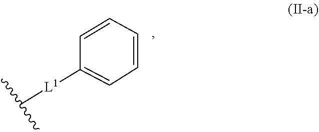
or
formula (III-a) 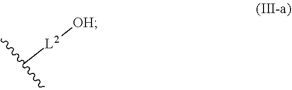
L¹ is C₁₋₄alkylene or C₁₋₂alkylene-O—C₁₋₂alkylene; and L² is C₁₋₄alkylene.

16. A composition comprising:
a plurality of block copolymers according to claim 1 self-assembled into a particle; and
a drug encapsulated within the particle.

17. The composition of claim 16, wherein the drug comprises a hydrogen bond acceptor moiety, an aromatic moiety, or a combination thereof.

18. The composition of claim 16, wherein the drug comprises a peptide-based drug, a chemotherapeutic, an anti-inflammatory drug, an immune modulating drug, or a combination thereof.

19. The composition of claim 16, wherein the drug comprises cyclosporin A, paclitaxel, bortezomib, etoposide, neratinib, osimertinib, chloroquine, GANT58, docetaxel, dexamethasone, carmofur, dexamethasone, dexamethasone palmitate, carfilzomib, afatinib, irinotecan, doxorubicin, doxycycline, camptothecin, imiquimod, MK-8617, ciclopirox, roxadustat, or a combination thereof.

20. The composition of claim 16, wherein the drug has a log D of about −1 to about 10.

21. The composition of claim 16, wherein the particle comprises the drug at about 0.5% to about 60% by weight of the particle.

22. The composition of claim 16, wherein the particle has a diameter of about 10 nm to about 500 nm.

23. A method of treating a disease or disorder in a subject in need thereof, the method comprising administering to the subject a therapeutically effective amount of the composition of claim 16.

24. The method of claim 23, wherein the disease or disorder is cancer, osteoarthritis, rheumatoid arthritis, ulcerative colitis, or wound healing.

25. The method of claim 23, wherein the composition has a maximum tolerated dose of at least 150 mg/kg.
